(19) United States
(12) Patent Application Publication
(10) Pub. No.: US 2026/0207936 A1
(43) Pub. Date: Jul. 23, 2026

(54) MULTI-NETWORK NEUROMODULATION

(71) Applicant: Otago Innovation Limited, Dunedin (NZ)

(72) Inventors: Dirk DE RIDDER, Dunedin (NZ); Divya Bharatkumar ADHIA, Dunedin (NZ); Paul William GLUE, Dunedin (NZ)

(73) Assignee: Otago Innovation Limited, Dunedin (NZ)

(21) Appl. No.: 19/157,884

(22) PCT Filed: Feb. 20, 2024
(86) PCT No.: PCT/NZ2024/050018

§ 371 Date: Aug. 19, 2025

Related U.S. Application Data
(60) Provisional application No. 63/447,025, filed on Feb. 20, 2023.

(51) Int. Cl. A61N 1/36; A61N 1/04

(52) U.S. Cl. CPC A61N 1/36025; A61N 1/0408; A61N 1/36034

(57) ABSTRACT

A therapeutic method in which low frequency wave forms are applied to targeted networks of interacting brain regions suffering from abnormal communication connections, particularly the central executive network, the default node network, and/or the salience network, to reinstate a normal correlated or an anticorrelated communication connection within and between such networks, and thereby treat brain-related disorders. This can be extended to four or five or more networks.

### FIELD OF THE INVENTION

The present invention relates to a neurostimulation method which can be utilized to treat brain-related conditions and/or disorders, and/or to ameliorate brain-related conditions, disorders or states.

More particularly, and not by way of limitation, the present invention is directed to a method for using one or more stimulation signals or waves in order to normalize connectivity between disrupted brain networks, to treat brain-related conditions and/or disorders and/or to ameliorate brain-related states.

### SUMMARY OF THE INVENTION

A non-invasive method of treating a brain-related disorder in a patient in need thereof may include ameliorating an abnormal condition within a first brain network and ameliorating one or more abnormal interactions between the first brain network and one or more further brain networks. The method may include delivering a first infraslow or slow stimulation wave to the first brain network. A second infraslow or slow stimulation wave may be delivered to at least one of the one or more further brain networks. A phase difference between the first and second infraslow or slow stimulation waves may be selected for amelioration of the abnormal interaction.

A non-invasive method of treating a brain-related disorder in a patient in need thereof may include ameliorating one or more abnormal interactions between a first brain network and one or more further brain networks. A first infraslow or slow stimulation wave may be delivered to the first brain network. A second infraslow or slow stimulation wave may be delivered to at least one of the one or more further brain networks. A phase difference between the first and second infraslow or slow stimulation waves is selected for amelioration of the abnormal interaction.

A non-invasive method of ameliorating a brain-related state in a patient may include ameliorating one or more interactions between a first brain network and one or more further brain networks. A first infraslow or slow stimulation wave may be delivered to the first brain network. A second infraslow or slow stimulation wave may be delivered to at least one of the one or more further brain networks. A phase difference between the first and second infraslow or slow stimulation waves is selected for amelioration of the brain-related state.

One or more further infraslow or slow stimulation waves may be delivered to one or more of the further brain networks.

At least one of the first and second stimulation waves may include a high frequency waveform nested upon an infraslow or slow carrier waveform.

The high frequency waveform may include one or more of: pink noise, brown noise, red noise, black noise, grey noise, white noise, blue noise, violet noise, or green noise.

The high frequency waveform may be defined as 1/f^{α}, with f being wave frequency and a being any number between −5 and +5, or is defined as a composite of 1/f^{α} and 1/f^{-α}, with f being wave frequency and a being any number between −5 and +5.

The high frequency waveform may be generated in a pseudo-random manner.

High-frequency waveform parameters may be set that define the high frequency waveform and at least one of said carrier waveform and said high frequency waveform may be defined to correspond to physiologic neural oscillations associated with at least one of: said first brain network and said one or more further brain networks.

The first brain network and one or more further brain networks may include the central executive network, the default node network and the salience network.

The one or more further brain networks may include a fourth network selected from the ventral attention network, dorsal attention network, memory network, emotion/affective network, emotional network, mirror neuron network, auditory network, visual network, somatosensory network, vestibular network, motor network, central autonomic nervous system network, central autonomic (control) network, sensorimotor network, and speech network.

The one or more further brain networks may include a fourth and fifth network selected from: ventral attention network, dorsal attention network, memory network, emotion/affective network, emotional network, mirror neuron network, auditory network, visual network, somatosensory network, vestibular network, motor network, central autonomic nervous system network, central autonomic (control) network, sensorimotor network, and speech network.

The method may include determining the presence of an abnormal correlation interaction or an abnormal anticorrelation interaction between the first brain network and at least one of the one or more further brain networks.

The method may include recording electrical activity related to neural activity in the brain of said patient; and, with reference to said recording, determining the presence of an abnormal correlation interaction or an abnormal anticorrelation interaction between the first brain network and at least one of the one or more further brain networks.

The method may include setting first waveform parameters that define the first infraslow or slow stimulation wave; and setting second waveform parameters that define the second infraslow or slow stimulation wave.

One or more stimulation wave generators may be configured to generate the first and second stimulation waves.

The abnormal interactions may include one or more abnormal correlation interactions and/or one or more abnormal anticorrelation interactions.

Ameliorating one or more abnormal interactions may include reinstating a normal correlated interaction. A normal correlated interaction may be reinstated by selecting the phase difference such that the first and second stimulation waves are in phase. A normal correlated interaction may be reinstated by delivering said first and second stimulation waves to the central executive network and the salience network, and the phase difference is such that the first and second stimulation waves are in phase.

Ameliorating one or more abnormal interactions may include reinstating a normal anticorrelated interaction. A normal anticorrelated interaction may be reinstated by selecting the phase difference such that the first and second stimulation waves are out of phase.

A normal anticorrelated interaction may be reinstated by delivering said first and second stimulation waves to the default mode network and the central executive network, and the phase difference is such that the first and second stimulation waves are out of phase.

A normal anticorrelated interaction may be reinstated by delivering said first and second stimulation waves to the default mode network and the salience network, and the phase difference is such that the first and second stimulation waves are out of phase.

The stimulation waveforms may be delivered to said patient contemporaneously.

The brain-related disorder or brain-related state may be characteristic of one or more of: disease attention deficit hyperactivity disorder, anxiety, depression, bipolar disorder, autism, obsessive compulsive disorder, post-traumatic stress disorder syndrome, or schizophrenia as well as mild cognitive impairment, dementias (Alzheimer, Lewy-body disease, multi-infarct) but also in thalamocortical dysrhythmias (tinnitus, pain, Parkinson Disease), stress, epilepsy, and disorders of consciousness (minimally cognitive state, vegetative state/unresponsive wakefulness syndrome), stress autonomic nervous system disorders including immune disorders.

A non-invasive neuromodulation system may be arranged to deliver stimulation waves trans-cranially to the brain of a patient, the system including: a first stimulation wave delivery unit, arranged to deliver a first infraslow or slow stimulation wave to a first brain network of the patient; and one or more further stimulation wave delivery units, each arranged to deliver a further infraslow or slow stimulation wave to a further brain network of the patient; and a controller arranged to control a phase difference between the first and second infraslow or slow stimulation waves, in order to ameliorate an abnormal interaction between the first brain network and at least one of the further brain networks.

A therapeutic method of treating a brain-related disorder in a patient in need of such treatment, may comprise determining the presence of an abnormal correlation interaction or an abnormal anticorrelation interaction between at least one or more nodes of a first identified network of interacting brain regions of interest in the patient with at least one or more nodes of one or more other identified networks of interacting brain regions of interest, and determining whether to reinstate a desired normal anticorrelation interaction therefore in the patient or a desired normal correlation interaction therefore in the patient. A practitioner may set first parameters that define a carrier waveform, wherein the carrier waveform exhibits an infraslow (0.0-0.1 Hz) or slow (0.1-1 Hz) selected waveform frequency. Then, the practitioner may set second parameters that define a high frequency waveform, that is nested upon the carrier waveform, and wherein at least one each of the carrier waveforms and the high frequency waveform are defined to correspond to physiologic neural oscillations that are known in the field to be associated with at least one of the identified networks of the interacting brain regions of interest. The practitioner may then provide one or more pulse generators that are configured to generate a plurality of nested stimulation electrical waveforms that are defined so as to promote or reinstate one or more desired normal correlation interactions and/or normal anticorrelation interactions in the patient. Promotion or reinstatement of normal anticorrelated and/or normal correlated interactions may be achieved in the claimed method of the invention by delivering a first nested stimulation waveform through one or more electrodes of the pulse generator or generators to the first identified network of interacting brain regions of interest, and then delivering a second nested stimulation waveform through one or more electrodes of the pulse generator or generators to the one or more other identified networks of interacting brain regions of interest in the patient.

This method can advantageously be extended to comprise the additional step of delivering at least a third or more nested stimulation waveforms through one or more electrodes of the pulse generators to a third or more other identified networks of interacting brain regions of interest in the patient.

In an alternative embodiment a method of treating a neurological, psychological or psychiatric disorder in a patient in need thereof may comprise identifying abnormal communication connectivity between at least two or more identified networks of interacting brain regions of interest in the patient, where the communication connectivity has been disrupted from a normal level of communication activity and that disruption is known in the field to be related to a certain disorder, then determining whether the disrupted communication connectivities are an abnormal communication connectivity correlation interaction or an abnormal communication connectivity anticorrelation interaction between a first identified network of interacting brain regions of interest in the patient with one or more other identified networks of interacting brain regions of interest. The practitioner may then set first parameters that define a carrier waveform, wherein the carrier waveform exhibits an infraslow (0.01-0.1 Hz) or slow (0.1-1 Hz) selected waveform, and then sets second parameters that define a high frequency waveform, which is nested upon the carrier waveform, and wherein at least one of the carrier waveform and the high frequency waveform are defined to correspond to physiologic (tonic, burst, noise) neural firing rates, local field potentials or oscillations associated with at least one of the networks of interacting brain regions of interest, then provides or obtains and operates at least one pulse generator which is configured so as to generate a plurality of the nested stimulation electrical waveforms, which individually are comprised of the carrier waveform and of the high frequency waveform, and are configured so as to reinstate one or more desired normal communication connectivity correlation interactions or normal communication connectivity anticorrelation interactions in the patient. Having set the pulse generator(s) configurations, the practitioner may then reinstate normal anticorrelated or normal correlated communication connectivity interactions by delivering to the patient a first nested stimulation waveform through one or more applied electrodes of the pulse generator to a first network of interacting brain regions of interest and by delivering a second nested stimulation waveform through one or more applied electrodes of the pulse generator to another identified network of interacting brain regions of interest.

There may be medical conditions in a patient where the patient is in need of being treated with a third or more additional other nested stimulation waveforms, and for such a patient the practitioner delivers a third or more nested stimulation waveforms through one or more applied electrodes of the pulse generator to a third or more additional other networks of interacting brain regions of interest in the patient.

In another alternative embodiment, the practitioner may engage the method of treating a neurological disorder in a patient, comprising the steps of identifying abnormal connectivity between at least two or more identified networks of interacting brain regions, where connectivity has been disrupted from the connectivity's normal communication activity, and the disruption is known in the field as being related to a brain-related disorder such as neurological, psychological, psychiatric, or autonomic disorder, recording electrical activity related to neural activity in the brain of the patient, and with reference to the recording, determining the presence of an abnormal correlation interaction or the presence of an abnormal anticorrelation interaction between each identified network of interacting brain regions, with each of the remaining other identified networks of interacting brain regions, and determining whether the patient needs to have the reinstatement of a normal anticorrelated or a normal correlated interaction. The practitioner may then set a first set of parameters that define a stimulation carrier waveform, wherein the carrier waveform exhibits an infraslow or slow selected waveform frequency in a range of frequencies of up to 1 Hz, and then sets a second set of parameters that define a stimulation high frequency waveform, where the stimulation high frequency (e.g. tonic, burst, noise) waveform is nested upon the stimulation carrier waveform, and wherein at least one of the stimulation carrier waveform and the stimulation high frequency waveform are defined to correspond to physiologic neural oscillations that are known in the field to be associated with at least one of the networks of the interacting brain regions. Having set the wave form parameters, the practitioner may then provide or obtain an electrical pulse generator to generate a plurality of nested stimulation electrical waveforms, which individually are comprised of the stimulation carrier waveforms and of the high frequency stimulation waveforms, wherein the nested stimulation waveform comprises a plurality of pulse bursts, and wherein the pulse bursts are characterized in that each of the pulse bursts is comprised of a plurality of discrete pulses, and furthermore that the plurality of discrete pulses within each pulse burst are repeated according to a frequency parameter of the high frequency waveform, and a wave amplitude of each of the discrete pulses, within respective such pulse bursts, is controlled according to the high frequency waveform nested on the carrier waveform such that wave amplitude peaks of the corresponding plurality of said discrete pulses vary within each respective one of the pulse burst(s). The practitioner may then reinstate normal anticorrelated or normal correlated interactions by delivering a first nested stimulation waveform through one or more applied pulse generator electrodes to a first network of interacting brain regions of interest, and delivering a second nested stimulation waveform through one or more applied pulse generator electrodes to a second network of interacting brain regions of interest.

There may be medical conditions in a patient where the patient is in need of being treated with a third or more additional other nested stimulation waveforms, and for such a patient the practitioner delivers a third or more nested stimulation waveforms through one or more applied electrodes of the pulse generator to a third or more additional other networks of interacting brain regions of interest in the patient.

Where a superimposed high frequency signal is used together with an infraslow or slow waveform, one or more of the nested stimulation electrical waveforms may be pink noise, brown noise, red noise, black noise, grey noise, white noise, blue noise, violet noise, or green noise. More particularly, the nested stimulation electrical waveforms may be quantitatively defined by the relationship 1/f^{α}, with f being wave frequency and a being any number between −5 and +5, or is defined as a composite of electrical waveforms of 1/f^{α} and 1/f^{-α}, with f being wave frequency and α being any number between −5 and +5.

The infraslow or slow waveform may have any suitable shape, including e.g. a squared or sinusoidal waveform.

As is known in the field, nesting of signals may take various forms, including e.g. cross-frequency coupling which can include coupling of power to power (or current density to current density), frequency to frequency, phase to phase, power to frequency, frequency to power, phase to frequency, frequency to phase, power to phase, phase to power. Such arrangements are discussed e.g. in Jensen, ‘Cross-frequency coupling between neuronal oscillations’, Trends in Cognitive Sciences Vol 11 No. 7, p 267.

The superimposed or nested signal can also be burst or tonic, i.e. not noisy, via cross-frequency coupling (phase-phase, amplitude-phase, power-power, current density-current density, phase-power, phase current density).

The nested noise stimulation electrical waveforms may be generated in a pseudo-random manner. In some embodiments of the invention, the nested noise stimulation waveforms may comprise pink noise nested on a carrier wave form. Alternatively, the nested noise stimulation waveforms may comprise grey noise nested on a carrier wave form.

The networks of interacting brain regions may be selected from the central executive network, the default node network, or the salience network. These networks may be further specifically targeted by said networks as the left and/or right central executive network, the left and/or right default node network, or the left and/or right salience network. Additionally, networks may be targeted in a strategy wherein the first identified network is targeted as though it is comprised of both the central network and the salience network, and the other identified network is comprised of the default mode network.

The method of the invention may target abnormal connectivity that communicates between the default mode network and the central executive network. Abnormal connectivity between at least the three networks of interacting brain regions described here is believed to be characteristic of disease attention deficit hyperactivity disorder, anxiety, depression, bipolar disorder, autism, obsessive compulsive disorder, post-traumatic stress disorder syndrome, or schizophrenia, as well as mild cognitive impairment, dementias (Alzheimer's disease, Lewy-body dementia, multi-infarct) but also in thalamocortical dysrhythmias (tinnitus, pain, Parkinson's disease), epilepsy, and disorders of consciousness (minimally cognitive state, vegetative state/unresponsive wakefulness syndrome).

Favorable outcomes may be achieved when an abnormal connectivity communication is restored to a normal anticorrelated communication between the central executive network and the salience network, or between the default mode network and the central executive network, or between the salience network and the default mode network.

Favorable outcomes can be achieved when an abnormal connectivity communication is restored to a normal correlated communication between the central executive network and the salience network, or between the default mode network and the central executive network, or between the salience network and the default mode network, or between any other suitable brain networks. Embodiments of the invention may contemporaneously deliver first, second, and third stimulation waveforms to a patient.

Furthermore, the phases of the wave forms, or phase differences between waveforms, can be configured to reinstate an abnormal connectivity communication to a normal anticorrelated communication by configuring an infraslow or slow carrier wave form to be opposite in phase between the default mode network and central executive network, or between the default mode network and the salience network, or between the central executive network and the salience network. Conversely, the phases of the wave forms, or phase differences between waveforms, can be configured to reinstate an abnormal connectivity communication to a normal correlated communication by configuring an infraslow or slow carrier wave form to be opposite in phase between the default mode network and central executive network, or between the default mode network and the salience network, or between the central executive network and the salience network.

The phases of the wave forms can conversely be configured to reinstate an abnormal connectivity communication to a normal anticorrelated communication by configuring an infraslow or slow carrier wave form to be in phase between the default mode network and the central executive network, or between the default mode network and the salience network, or between the central executive network and the salience network. Conversely, the phases of the wave forms can be configured to reinstate an abnormal connectivity communication to a normal correlated communication by configuring an infraslow or slow carrier wave form to be in phase between the default mode network and the central executive network, or between the default mode network and the salience network, or between the central executive network and the salience network.

In one embodiment of the invention as claimed, the patient may be treated with grey noise stimulation to achieve a normalized anticorrelation connectivity communication between the left central executive network, the left default mode network, and the right salience network. In another embodiment this may be performed bilaterally for all networks or any combination of left and right networks.

This invention relates to non-invasive methods of neurostimulation such as transcranial magnetic stimulation, transcranial electrical stimulation, transcranial ultrasound, transcranial optic (light or laser) stimulation, in all their different forms.

A method of treating a neurological disorder in a patient in need thereof, may comprise the steps of:
(a) determining the presence of an abnormal correlation interaction or an abnormal anticorrelation interaction between at least one or more nodes of a first identified network of interacting brain regions of interest with at least one or more nodes of one or more other identified networks of interacting brain regions of interest, and determining whether to reinstate a desired normal anticorrelation interaction therefore or a desired normal correlation interaction therefore;
(b) setting first parameters that define a carrier waveform, wherein said carrier waveform exhibits an infraslow or slow selected waveform frequency;
(c) setting second parameters that define a high frequency waveform, that is nested upon said carrier waveform, and wherein at least one of said carrier waveform and said high frequency waveform are defined to correspond to physiologic neural oscillations associated with at least one of said identified networks of interacting brain regions of interest;
(d) providing one or more pulse generators configured to generate a plurality of nested stimulation electrical waveforms defined to reinstate one or more desired normal correlation interactions or normal anticorrelation interactions in said patient;
(e) reinstating normal anticorrelated or normal correlated interactions by delivering a first nested stimulation waveform through one or more electrodes of said pulse generators to said first identified network of interacting brain regions of interest; and
(f) delivering a second nested stimulation waveform through one or more electrodes of said pulse generators to said one or more other identified networks of interacting brain regions of interest.

The method may comprise the additional step of delivering at least a third or more nested stimulation waveforms through one or more electrodes of said pulse generators to a third or more other identified networks of interacting brain regions of interest.

A method of treating a neurological disorder in a patient in need thereof may comprise the steps of:
(a) identifying abnormal communication connectivity between at least two or more identified networks of interacting brain regions of interest where said communication connectivity has been disrupted from a normal level of communication activity and said disruption is related to a neurological disorder;
(b) determining whether said disrupted communication connectivities are an abnormal communication connectivity correlation interaction or an abnormal communication connectivity anticorrelation interaction between a first identified network of interacting brain regions of interest with one or more other identified networks of interacting brain regions of interest;
(c) setting first parameters that define a carrier waveform, wherein said carrier waveform exhibits an infraslow or slow selected waveform frequency in a range of frequencies of up to 1 Hz;
(d) setting second parameters that define a high frequency waveform, that is nested upon said carrier waveform, and wherein at least one of said carrier waveform and said high frequency waveform are defined to correspond to physiologic neural oscillations associated with at least one of said networks of interacting brain regions of interest;
(e) providing and operating at least one pulse generator configured so as to generate a plurality of said nested stimulation electrical waveforms, which individually are comprised of said carrier waveform and of said high frequency waveform, and are configured to reinstate one or more desired normal communication connectivity correlation interactions or normal communication connectivity anticorrelation interactions in said patient;
(f) reinstating normal anticorrelated or normal correlated communication connectivity interactions by delivering a first nested stimulation waveform through one or more applied electrodes of said pulse generator to a first network of interacting brain regions of interest and by delivering a second nested stimulation waveform through one or more applied electrodes of said pulse generator to an other network of interacting brain regions of interest; and
(g) where said patient is in need of a third or more other nested stimulation waveforms, delivering a third or more nested stimulation waveforms through one or more applied electrodes of said pulse generator to a third or more additional other networks of interacting brain regions of interest.

A method of treating a neurological disorder in a patient may comprise the steps of:
(a) identifying abnormal connectivity between at least two or more identified networks of interacting brain regions where connectivity has been disrupted from its normal communication activity and said disruption is related to a neurological disorder;
(b) recording electrical activity related to neural activity in the brain of said patient;
(c) with reference to said recording, determining the presence of an abnormal correlation interaction or an abnormal anticorrelation interaction between each identified network of interacting brain regions with each of the remaining other identified networks of interacting brain regions, and determining whether to reinstate a normal anticorrelated or a normal correlated interaction;
(d) setting first parameters that define a stimulation carrier waveform, wherein said carrier waveform exhibits an infraslow or slow selected waveform frequency in a range of frequencies of up to 1 Hz;
(e) setting second parameters that define a stimulation high frequency waveform, where said high frequency waveform is nested upon said carrier waveform, and wherein at least one of said stimulation carrier waveform and said high frequency waveform are defined to correspond to physiologic neural oscillations associated with at least one of said networks of interacting brain regions;
(f) providing and operating a pulse generator to generate a plurality of nested stimulation electrical waveforms, which individually are comprised of said carrier waveforms and of said high frequency waveforms, wherein said nested stimulation waveform comprises a plurality of pulse bursts, and wherein said pulse bursts are characterized in that each of said pulse bursts is comprised of a plurality of discrete pulses, and said plurality of discrete pulses within each pulse burst are repeated according to a frequency parameter of said high frequency waveform, and wave amplitude of each said discrete pulse within respective said pulse bursts is controlled according to said high frequency waveform nested on said carrier waveform such that wave amplitude peaks of the corresponding plurality of said discrete pulses vary within each respective said pulse burst;
(g) reinstating normal anticorrelated or normal correlated interactions by delivering a first nested stimulation waveform through one or more applied pulse generator electrodes to a first network of interacting brain regions of interest, and delivering a second nested stimulation waveform through one or more applied pulse generator electrodes to a second network of interacting brain regions of interest; and
(g) where said patient is in need of a third or more other nested stimulation waveforms, delivering a third or more nested stimulation waveforms through one or more applied electrodes of said pulse generator to a third or more additional other networks of interacting brain regions of interest.

One or more of said nested stimulation electrical waveforms may be pink noise, brown noise, red noise, black noise, grey noise, white noise, blue noise, violet noise, or green noise.

One or more of said nested stimulation electrical waveforms may be defined as 1/f, with f being wave frequency and α being any number between −5 and +5, or is defined as a composite of 1/f^{α} and 1/f^{-α}, with f being wave frequency and α being any number between −5 and +5.

The nested noise stimulation electrical waveform may be generated in a pseudo-random manner.

One or more of said nested noise stimulation waveforms may comprise pink noise nested on a carrier wave form.

One or more of said nested noise stimulation waveforms may comprise grey noise nested on a carrier wave form.

Said networks of interacting brain regions may be selected from the central executive network, the default node network, or the salience network.

Said abnormal connectivity between at least three networks of interacting brain regions may be characteristic of disease attention deficit hyperactivity disorder, anxiety, depression, bipolar disorder, autism, obsessive compulsive disorder, post-traumatic stress disorder syndrome, or schizophrenia as well as mild cognitive impairment, dementias (Alzheimer's disease, Lewy-body dementia, multi-infarct) but also in thalamocortical dysrhythmias (tinnitus, pain, Parkinson's disease), stress, epilepsy, and disorders of consciousness (minimally cognitive state, vegetative state/unresponsive wakefulness syndrome), stress autonomic nervous system disorders including immune disorders.

Said abnormal connectivity may communicate between said default mode network and said central executive network.

Said abnormal connectivity communication may be reinstated to a normal anticorrelated communication.

Said abnormal connectivity may communicate between said central executive network and said salience network.

Said abnormal connectivity communication may be reinstated to a normal correlated communication.

Said abnormal connectivity may communicate between said salience network and said default mode network.

Said abnormal connectivity communication may be reinstated to a normal anticorrelated communication.

Said first, second, and third nested stimulation waveforms may be delivered to said patient contemporaneously.

Said abnormal connectivity communication may be reinstated to a normal anticorrelated communication by configuring said infraslow or slow carrier wave form to be opposite in phase between said default mode network and said central executive network.

Said abnormal connectivity communication may be reinstated to a normal anticorrelated communication by configuring said infraslow or slow carrier wave form to be opposite in phase between said default mode network and said salience network.

Said abnormal connectivity communication may be reinstated to a normal anticorrelated communication by configuring said infraslow or slow carrier wave form to be opposite in phase between said central executive network and said salience network.

Said abnormal connectivity communication may be reinstated to a normal anticorrelated communication by configuring said infraslow or slow carrier wave form to be in phase between said default mode network and said central executive network.

Said abnormal connectivity communication may be reinstated to a normal anticorrelated communication by configuring said infraslow or slow carrier wave form to be in phase between said default mode network and said salience network.

Said abnormal connectivity communication may be reinstated to a normal anticorrelated communication by configuring said infraslow or slow carrier wave form to be in phase between said central executive network and said salience network.

Said first identified network may be comprised of the central network and the salience network, and the other identified network may be comprised of the default mode network.

A neurostimulator device for treating a neurological disorder in a patient in need thereof, may be configured as to execute the steps of:
(a) determining the presence of an abnormal correlation interaction or an abnormal anticorrelation interaction between at least one or more nodes of a first identified network of interacting brain regions of interest with at least one or more nodes of one or more other identified networks of interacting brain regions of interest, and determining whether to reinstate a desired normal anticorrelation interaction therefore or a desired normal correlation interaction therefore;
(b) setting first parameters that define a carrier waveform, wherein said carrier waveform exhibits an infraslow or slow selected waveform frequency;
(c) setting second parameters that define a high frequency waveform, that is nested upon said carrier waveform, and wherein at least one of said carrier waveform and said high frequency waveform are defined to correspond to physiologic neural oscillations associated with at least one of said identified networks of interacting brain regions of interest;
(d) providing one or more pulse generators configured to generate a plurality of nested stimulation electrical waveforms defined to reinstate one or more desired normal correlation interactions or normal anticorrelation interactions in said patient;
(e) reinstating normal anticorrelated or normal correlated interactions by delivering a first nested stimulation waveform through one or more electrodes of said pulse generators to said first identified network of interacting brain regions of interest; and
(f) delivering a second nested stimulation waveform through one or more electrodes of said pulse generators to said one or more other identified networks of interacting brain regions of interest.

### BACKGROUND OF THE INVENTION

Neurostimulation or neurological stimulation (NS) systems are devices that generate stimulation waves (e.g. waveforms including pulses or other waves) and deliver the waves to nervous tissue to treat a variety of disorders.

Recently, new stimulation configurations such as burst stimulation and high frequency stimulation have been developed, in which closely spaced high frequency pulses are delivered. In general, conventional neurostimulation systems seek to manage pain and other pathologic or physiologic disorders through stimulation of select nerve fibers that carry pain related signals. However, nerve fibers and brain tissue carry other types of signals, not simply pain related signals.

Although some neurological disorders have been treated through known neurostimulation methods, many other neurological disorders exhibit physiological complexity, functional complexity, or other complexity and have not been adequately treated through known neurostimulation methods. Current techniques in brain stimulation are also largely based on a phrenological approach that a single brain target can treat a brain disorder.

### BRIEF DESCRIPTION OF THE FIGURES

The invention will be described by way of example only, with reference to the drawings, in which:

FIG. 1A illustrates altered connectivity involving the central executive network.

FIG. 1B illustrates altered connectivity involving the default mode network.

FIG. 2 illustrates hypoconnectivity and hyperconnectivity between brain networks.

FIG. 3 shows a stimulation wave according to one embodiment, including an infraslow or slow carrier waveform with a nested or superimposed noise waveform. This figure also illustrates various types of noise waveform.

FIG. 4 illustrates correlation and anticorrelation between brain networks.

FIG. 5 shows how stimulation waves may be applied to brain networks to ameliorate abnormal correlation or anticorrelation conditions. Normalization of triple network interactions via infraslow stimulation may be combined with pink noise nested on top of the infraslow stimulus. The infraslow component permits to correlate activity within networks and between the salience and central executive network. It also permits to anticorrelate activity between the default mode and the salience and central executive network by supplying infraslow stimuli in antiphase at the default mode and the salience plus central executive network. The pink noise nested on top of this infraslow component serves to mimic normal physiological brain activity.

FIG. 6 illustrates expected outcomes with a delayed start study protocol.

FIG. 7 shows results from a study based on the Montgomery-Asberg Depression Rating Scale (MADRS), showing the improvement of depression symptoms with treatment over placebo. On this scale, 0-6 is considered normal, no symptoms; 7-19 is considered mild depression; 20-34 is considered moderate depression; over 34 is considered severe depression.

FIG. 8 shows results from a study based on a Quick Inventory of Depressive Symptomatology (QIDS) self-report. On this scale, ≤5 represents no depression; 6-10 is considered mild depression; 11-15 is considered moderate depression; 16-20 is considered severe depression and ≥21 is considered very severe depression.

FIG. 9 show results from a study based on the Ruminative Response Scale (RRS).

FIG. 10 shows a questionnaire used in the WHO-5 Well Being Index.

FIG. 11 shows the results of a study based on the WHO-5 Well Being Index.

FIGS. 12 and 13 show the results of a study based on the Hamilton Anxiety and Depression Scale (HADS) On this scale, 0-7 represents no anxiety, no depression; 8-10 indicates mild symptoms; 11-14 indicates moderate symptoms; and 15-21 indicates severe symptoms.

FIGS. 14 and 15 show the effect of the level of noise on functional connectivity. Stochastic resonance effect of noise stimulation can both increase and decrease connectivity. With low amplitudes no change in connectivity ensues as the firing threshold is not reached (weak noise in FIG. 14). Optimal amplitudes push activity at different areas above threshold, leading to increased connectivity (optimal noise FIG. 14). High amplitudes create noise at the two areas, preventing phase synchronization and thus functional connectivity (high noise FIG. 14). This creates an inverted U curve profile in connectivity (FIG. 15).

FIG. 16 shows a method acting on four networks, such as might be used in treating, for example, Anxiety, depression, PTSD or schizophrenia.

FIG. 17 illustrates a method acting on three networks plus two examples of a fourth network, for treating pain or tinnitus.

FIG. 18 illustrates one example of a neuromodulation system.

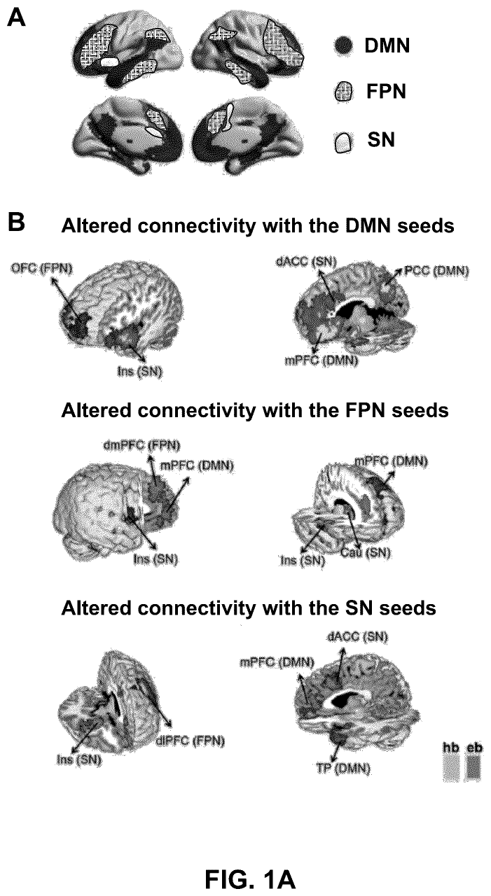

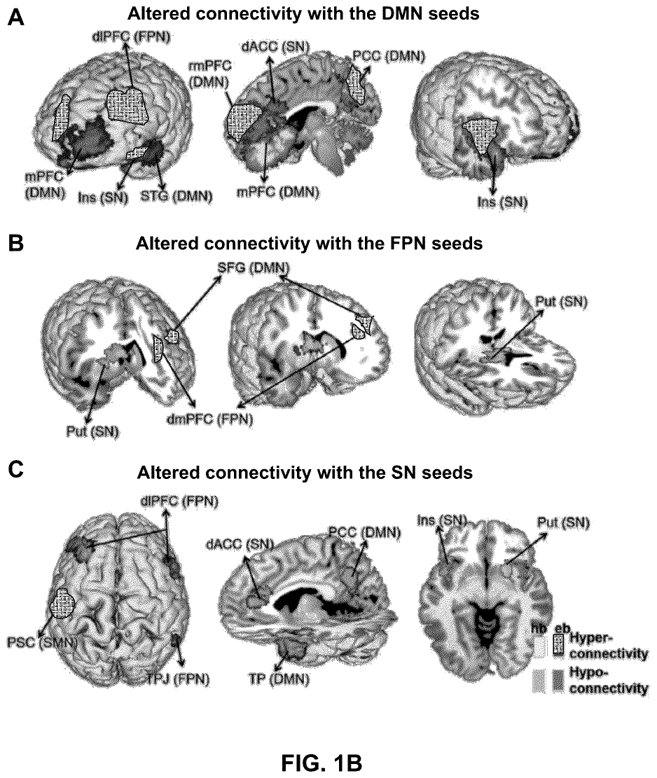

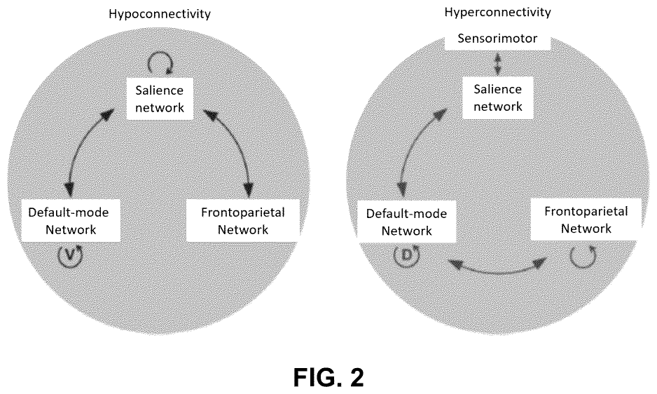

### DETAILED DESCRIPTION OF THE INVENTION AND OF THE FIGURES

While multiple embodiments are described, still other embodiments of the described subject matter will become apparent to those skilled in the art from learning from the following detailed novel disclosure descriptions and drawings, which show and describe illustrative embodiments of disclosed inventive subject matter as claimed. As will be realized, the inventive subject matter is capable of modifications in various aspects, all without departing from the spirit and scope of the described subject matter. Accordingly, the drawings and detailed description are to be regarded as illustrative in nature and not restrictive.

Definitions. Wherever used herein, the usage of the word “a” or “an”, when used in conjunction with the term “comprising” in the claims and/or the specification, shall mean “one,” or “one or more,” or “at least one,” or “one or more than one.” The terms “having”, “including”, “containing”, and “comprising” are interchangeable and one of skill in the art may readily recognize that these terms are open-ended terms. If a term is not defined in this specification, then technical and scientific terms used herein have the same meaning as commonly understood by one of ordinary skill in the art to which this invention belongs. For purposes of the descriptions of the invention, the following terms are defined below.

Action Potential. An action potential is a depolarization that occurs when the membrane potential of a specific cell location rapidly rises and falls; membrane potential here means the difference in electric potential between the interior and the exterior of a biological cell. The terms “pulse” and “spike” are used interchangeably herein to refer to an action potential.

Alpha. A brainwave neural oscillation falling into a frequency band in the range of from about 8 to about 12 Hz.

Beta. A brainwave neural oscillation falling into a frequency band in the range of from about 13 to about 30 Hz.

Brain networks. Wherever a brain network is named as an element of the method or devices of the invention, the network shall be understood and interpreted to be describing and claiming each region, subregion, system or subsystem of such network, both in the anatomical sense thereof, and in the functional sense thereof, all as known to those of ordinary skill in the art. However, at minimum a brain network includes two or more interconnected nodes with a correlated activity pattern.

Brain-related disorder: any illness, disease or disorder than can be linked to abnormal functioning of the brain. This encompasses mental, psychological or psychiatric disorders, but also neurological, autonomic nervous system disorders, as well as immune disorders, endocrinological disorders, and more generally any pathology that is under control of the nervous system.

Burst: “Burst” refers to a period in a spike train that has a much higher discharge rate than surrounding periods in the spike train (N. Urbain et. al., 2002) Thus, burst can refer to a plurality of groups of spike pulses A burst is a train of action potentials that, possibly, occurs during a ‘plateau’ or ‘active phase’, followed by a period of relative quiescence called the ‘silent phase’ (Nunemaker, Cellscience Reviews Vol 2 No. 1, 2005.) Thus, a burst comprises spikes having an inter-spike interval in which the spikes are separated by about or approximately 0.5 milliseconds, or a time period that is functionally equivalent, to about or approximately 100 milliseconds, or a time period that is functionally equivalent. Those of skill in the art realize that the inter-spike interval can be longer or shorter. Yet further, those of skill in the art also realize that the spike rate within the burst does not necessarily occur at a fixed rate; this rate can be variable.

Burst Firing or Burst Mode: Burst mode refers to an action potential that is a burst of high frequency spikes (e.g. in the range of about 400-1000 Hz) (Beurrier et al., 1999). Burst firing acts in a non-linear fashion with a summation effect of each spike. One skilled in the art is also aware that burst firing can also be referred to as phasic tiring, rhythmic firing (Lee 2001) pulse train firing, oscillatory firing and spike train firing, and all of these terms as may be used herein are interchangeable.

Burst Spike. A “burst spike” refers to a spike that is preceded or followed by another spike within a short time interval (Matveev, 2000), in other words, there is an inter-spike interval, in which this interval is generally about 100 ms but can be shorter or longer, for example 0.5 milliseconds.

Central Executive Network (CEN). The central executive network (CEN), generally also known among those of ordinary skill in the art as the frontoparietal control network, frontoparietal network (FPN) or, more specifically, the lateral frontoparietal network (L-FPN), is a large-scale brain network primarily composed of the dorsolateral prefrontal cortex and the posterior parietal cortex around the intraparietal sulcus. It is involved in cognitive functioning, complex problem-solving and working memory. The CEN is one of three networks referred to in the field as the so-called triple-network model, along with the salience network (SN) and the default mode network (DMN). The salience network facilitates switching between the CEN and DMN. The CEN is primarily composed of the rostral lateral and dorsolateral prefrontal cortex (especially the middle frontal gyrus) and the anterior inferior parietal lobule. Additional regions include the middle cingulate gyrus and potentially the dorsal precuneus, posterior inferior temporal lobe, dorsomedial thalamus and the head of the caudate nucleus.

Central Nervous System: The central nervous system (CNS) comprises the brain and spinal cord, which together function as the principal integrator of sensory input and motor output. In general terms, the brain consists of the cerebrum (cerebral hemispheres and the diencephalons), the brainstem (midbrain, pons, and medulla); and the cerebellum. It is well known that the cerebrum represents the highest center for sensory and motor and emotional and cognitive processing. In general, the frontal lobe processes motor, visual, speech, and personality modalities; the parietal lobe processes sensory information; the temporal lobe, auditory and memory modalities; and the occipital lobe vision. The cerebellum, in general, coordinates smooth motor activities and processes muscle position, while the brainstem conveys motor and sensory information and mediates important autonomic functions. These structures are of course integrated with the spinal cord which receives sensory input from the body and conveys somatic and autonomic motor information to peripheral targets. Thus, one of skill in the art realizes that the central nervous system is capable of evaluating incoming information and formulating response to changes that threaten the homeostasis of the individual.

Central neuronal tissue: “Central neuronal tissue” refers to neuronal tissue associated with the brain, spinal cord or brainstem.

Delta: A brainwave frequency band falling into the range of from 1 to 4 Hz.

Disorder: a functional abnormality or disturbance, can be used interchangeably with illness, disease, or pathology

Electroencephalograph: a measurement, detected and displayed by electronic devices well known to those of ordinary skill in the art, of electrical impulses that are generated by collective brain neurons. Such impulses are identifiable as brainwaves falling into different ranges of frequencies. Electrodes connected to the device are placed on specific sites on the scalp to detect and record the electrical impulses that collections of neurons are generating within the brain.

Frequency: The number of times a wave repeats itself within a second.

Frontoparietal network. The frontoparietal network (FPN), generally also known among those of ordinary skill in the art as the central executive network (CEN) or, more specifically, the lateral frontoparietal network (L-FPN), is a large-scale brain network primarily composed of the dorsolateral prefrontal cortex and posterior parietal cortex around the intraparietal sulcus. It is involved in sustained attention, complex problem-solving and working memory. The FPN is one of three networks referred to in the field as the so-called triple-network model, along with the salience network (SN) and the default mode network (DMN). The salience network facilitates switching between the FPN and DMN. The FPN is primarily composed of the rostral lateral and dorsolateral prefrontal cortex (especially the middle frontal gyrus) and the anterior inferior parietal lobule. Additional regions include the middle cingulate gyrus and potentially the dorsal precuneus, posterior inferior temporal lobe, dorsomedial thalamus and the head of the caudate nucleus.

Functional Magnetic Resonance Imaging (fMRI).

Gamma. A brainwave frequency band falling into the range of greater than 30 Hz, typically though not exclusively up to 100 Hz.

Hz: Hertz, the unit of measurement of wave frequency, defined as one cycle per second.

Infraslow. A brainwave frequency band falling into the range of less than 0.1 Hz.

Medial Frontoparietal Network (M-FPN). The functional network that is the Default Mode Network (DMN) is known anatomically as the Medial Frontoparietal Network.

Modulate: To modulate refers to the ability to regulate positively or negatively neuronal activity. Thus, the term modulate can be used to refer to an increase, decrease, masking, altering, overriding, restoration, phase reversal, correlation alteration, correlation restoration, anticorrelation alteration, or anticorrelation restoration of neuronal activity.

Neuroenhancement: ameliorating non-pathological brain-related states, for example to improve cognition, improve sports peak performance, or strengthen resilience

Neurology or Neurological: “Neurology” or “neurological” refers to conditions, disorders, and/or diseases that are associated with the nervous system. The nervous system comprises two components, the central nervous system, which is composed of the brain and the spinal cord, and the peripheral nervous system, which is composed of ganglia and the peripheral nerves that lie outside the brain and the spinal cord. As used herein, the term “neurological” or “neurology” encompasses the terms “neuropsychiatric” or “neuropsychiatry” and “neuropsychological” or “neuropsychology”. Thus, for example, a neurological disease, condition, or disorder includes, but is not limited to, tinnitus, epilepsy, depression, anxiety, Parkinson's Disease, or autonomic dysfunctions, and the like.

Neuromodulation: Neuromodulation is “the alteration of nerve activity through targeted delivery of a stimulus, such as magnetic, electrical, light, sound stimulation or chemical agents, to specific neurological sites in the body”. It is carried out to improve or normalize—or modulate—nervous tissue function. Neuromodulation, whether electrical or magnetic, employs the body's natural biological response by stimulating nerve cell activity that can influence populations of nerves by releasing neurotransmitters, such as dopamine, serotonin and others or other chemical messengers such as the neuropeptides, that can modulate the excitability and firing patterns of neural circuits. There may also be more direct electrophysiological effects on neural membranes as the mechanism of action of electrical interaction with neural elements. The end effect is an improvement or “normalization” of a neural network function from its perturbed state. Presumed mechanisms of action for neurostimulation include depolarizing blockade, stochastic normalization of neural firing, axonal blockade, reduction of neural firing keratosis, and suppression of neural network oscillations.

Neuronal: “Neuronal” refers to a cell type which is a morphologic and functional unit of the brain, brainstem, spinal cord, and/or peripheral nerves.

Neuropsychiatry or Neuropsychiatric: “Neuropsychiatry” or “neuropsychiatric” refers to conditions, disorders and/or diseases that relate to either or both organic and psychic/mental disorders of the nervous system.

Neuropsychological or Neuropsychologic: “Neuropsychological” or “neuropsychologic” or “neuropsychology” refers to conditions, disorders and/or any disease that relates to the functioning of the brain and the cognitive processes, or processors, or behavior.

Neurostimulation: Neurostimulation is the purposeful modulation of the nervous system's electrical activity using invasive (e.g. surgically implanted microelectrodes) or using non-invasive means. This embodiment of the invention uses non-invasive means. Neurostimulation shall refer to the electromagnetic approaches to neuromodulation. It can be used interchangeably with neuromodulation.

Orbitofrontal cortex (OFC). The orbitofrontal cortex (OFC) is a prefrontal cortex region in the frontal lobes of the brain, which is involved in the cognitive process of decision-making. In humans it consists of Brodman areas 10, 11, and 47. The OFC is functionally related to the ventromedial prefrontal cortex. Therefore, the region is distinguished due to the distinct neural connections and the distinct functions it performs. It is defined as the part of the prefrontal cortex that receives projections from the medial dorsal nucleus of the thalamus, and is thought to represent emotion, taste, olfaction and the action of reward in decision making. It gets its name from its position immediately above the orbits in which the eyes are located.

Peripheral neuronal tissue: “Peripheral neuronal tissue” refers to any neuronal tissue associated with a nerve root, root ganglion, or peripheral nerve that is outside the brain and the spinal cord, including the autonomous nervous system, inclusive of (ortho) sympathetic and parasympathetic systems, and thus the term non-peripheral neuronal tissues excludes these categories.

Phase difference: a difference in phase between two stimulation waves. Waves may be generally in phase (having a phase difference of zero, or approximately zero), generally out of phase (phase difference 180 degrees, or approximately 180 degrees), or have some other phase difference. As an example, for reinstatement of an anticorrelated state, a phase difference of around 180 degrees, or between 135 and 225, or between 90 and 270 degrees, may be used. As a further example, for reinstatement of a correlated state, a phase difference of around 0 degrees, or between 315 and 45 degrees, or between 270 and 90 degrees, may be used. In general, this specification is concerned with phase differences between infraslow or slow stimulation waves.

Psychiatric condition or disorder: a behavioral or mental pattern that causes significant distress or impairment of personal functioning; It can be used interchangeably with psychological or mental disorder.

Psychological condition or disorder: a psychological pattern associated with distress or disability that occurs in a person and is not a part of normal development or culture. It can be used interchangeably with psychiatric or mental disorder.

Sigma: A brainwave frequency band falling into the range of greater than 500 Hz.

Slow (wave): A brainwave frequency band falling into the range of 0.1 to 1 Hz.

Spike: “spike” refers to an action potential. Yet further, a “burst spike” refers to a spike that is preceded or followed by another spike within a short time interval (Matveev, 2000), in other words, there is an inter-spike interval, in which this interval is generally about 100 ins but can be shorter or longer, for example 0.5 milliseconds.

Stimulation: Stimulation, as used herein, refers to electrical, chemical, magnetic, thermal, sound and/or other such stimulation that modulates the predetermined neuronal sites, and in this application shall largely refer to neurostimulation.

Theta. A brainwave frequency band falling into the range of from 4 to 8 Hz.

Tonic Firing or Tonic Mode. “Tonic firing” or “tonic mode” refers to an action potential that occurs in a linear fashion.

Transcranial Pulse Stimulation (TPS). A non-invasive neurostimulation method that uses pulses delivered to the brain from a source external to the cranium.

Treating and Treatment. “Treating” and “treatment” refer to modulating predetermined neuronal sites, particularly central neuronal tissue, so that the subject has an improvement in the disease or condition, for example, beneficial or desired clinical results. For purposes of this invention, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease, which will be unpredictable unknowable until clinical testing and observation of a novel treatment have been completed.

Action Potentials and Their Propagation. Information is conveyed through the nervous system via neuronal cells along their membranes and across synaptic junctions. Thus, the neuronal cells process information by both passive processes (e.g., electrical properties of the membrane which enable spatial and temporal summation) and active processes (e.g., propagation of the action potential, signal amplification or attenuation, and synaptic transmission). Generation of an action potential at the axon initial segment requires passive summation of multiple inputs, as well as signal amplification before membrane depolarization reaches threshold, thus the passive and active processes are interdependent.

The generation of the action potential initially depends upon the electrical properties of the cell. It is known that cells have an electrical voltage difference across their membranes, the membrane potential. Several types of protein pores or ion channels are responsible for maintaining and altering the membrane potential of the cell. Voltage-gated sodium channels, which have a low threshold, are responsible for the explosive depolarization of the membrane potential that forms the action potential or spike, whereas, the voltage-gated potassium channels are responsible for the repolarization of the membrane potential. For excitation, stimulatory input results in a net increase in the inward flow of sodium ions compared to an outward flow of potassium ions, which results in a depolarizing cell membrane potential change. For inhibitory inputs, potassium and chloride ion channels are opened which drives the membrane potential away from threshold (hyperpolarization).

As one of skill in the art realizes, neurons receive multiple excitatory and inhibitory inputs, thus a summation of these inputs occurs, for example temporal and spatial summations. Temporal summation occurs when a series of subthreshold impulses in one excitatory fiber produces an action potential in a postsynaptic cell. Spatial summation occurs when subthreshold impulses from two or more different fibers trigger an action potential.

Once the initial action potential is generated, the information is conveyed via axonal conduction or synaptic transmission (e.g., chemical or electrical). Electrical synapses are found not only in the brain, but in heart and smooth muscle and epithelial liver cells. However, in the brain, electrical synapses (also known as gap junctions) are less common than chemical synapses, and are characterized by rapid speed of transmission and do not readily allow inhibitory actions or long-lasting changes in effectiveness. Gap junctions allow the passage of not only ions, but other small molecules. In humans, astrocytes contain gap junctions to mediate potassium buffering.

Chemical synapses, on the contrary, do mediate either excitatory or inhibitory actions, and are generally considered more flexible. Another difference between chemical and electrical transmission is that electrical can be bidirectional since the ion channels connect the cytoplasm of the presynaptic and the postsynaptic cells, whereas chemical transmission is typically unidirectional since there is no continuity between the cells. Chemical synapses comprise a presynaptic element that contain vesicles comprising neurotransmitters and a postsynaptic element which contains receptors for the neurotransmitters. Transmitter release is initiated when the nerve terminal is depolarized by an action potential resulting in a rapid influx of calcium ions into the nerve terminal. This rapid influx of calcium ions causes a fusion of the vesicles to the presynaptic membrane, and ultimately the release of the neurotransmitters, which then bind to their receptor located on the postsynaptic membrane.

The ability of the neuronal cell to fire or produce action potentials may vary depending upon its biophysical properties (e.g., types of ionic channels, etc.) and/or its position in the circuit or nervous system. Thus, cells can respond to an input (stimulatory or inhibitory) with a decelerating train of action potentials, an accelerating train of action potentials or a constant firing frequency. For example, an increase in firing of a neuronal cell may be a result from increased amounts of calcium ions or a function of residual increase in calcium ions left over from the first stimulation (also known as facilitation) in the presynaptic element, which results in an increased release of the neurotransmitter. Thus, a second stimulation can occur within milliseconds of the first. Conversely, a second stimulation may result in inhibition and not facilitation of the response if an inhibitory interneuron is activated, which feedbacks to the first neuronal cell to inhibit firing.

### Brain Network Connectivity and Stimulation Patterns.

Default Mode Network (DMN). The default mode network (DMN) is a network of interacting brain regions that is active when a person is not focused on the person's outside world, and is measurable by using the fMRI technique. In the field of neuroscience, the DMN may also be known as the default network, the default state network, or anatomically as the medial frontoparietal network (M-FPN), and is generally described as a large-scale brain network that is primarily composed of the medial prefrontal cortex, the posterior cingulate cortex/precuneus, or the angular gyrus. The DMN is best known for being active when a person is not focused on the outside world and the brain is at wakeful rest, such as during daydreaming and mind-wandering. It may also be active during detailed thoughts related to external task performance. Other times that the DMN is active include such times as when the individual is thinking about others, thinking about themselves, remembering the past, or planning for the future. The DMN was originally noticed to be deactivated in certain goal-oriented tasks and was sometimes referred to as the task-negative network, contrasting with the task-positive network. This nomenclature is not preferred, because it is now known that the network can be active in internal goal-oriented and conceptual cognitive tasks. The DMN has been shown to be negatively correlated with other networks in the brain such as attention networks.

The brain, however is constantly busy and does not cease activity when a subject is at rest. Metabolism in the brain stays the same when a person goes from a resting state to performing math problems requiring mental effort, suggesting that active metabolism in the brain must also be happening during rest. In fact, the brain's energy consumption is increased by less than 5% of its baseline energy consumption while performing a focused mental task. This shows that the brain is constantly active with a high level of activity even when the person is not engaged in focused mental work. Raichle coined the term “default mode” in 2001 to describe resting state brain function; the concept rapidly became a central theme in neuroscience. Around this time the idea was developed that this network of brain areas is involved in internally directed thoughts and is suspended during specific goal-directed behaviors. In 2003, Greicius and colleagues examined resting state fMRI scans and looked at how correlated different sections in the brain are to each other, creating correlation maps. Since then, other networks have been identified, such as visual, auditory, and attention networks, and some of them are often anti-correlated with the default mode network.

The default mode network is thought to be involved in several different functions. It is potentially the neurological basis for the self, when it engages in autobiographical information. i.e. memories of collection of events and facts about one's self; self-reference, that is, referring to traits and descriptions of one's self; and emotion of one's self, which is the practice of reflecting about one's own emotional state.

It is potentially the neurological basis for thinking about others, including the theory of mind-thinking about the thoughts of others and what they might or might not know; the emotions of others, in understanding the emotions of other people and empathizing with their feelings; in moral reasoning, determining a just and an unjust result of an action; social evaluations for example forming good-bad attitude judgments about social concepts; in social categories, as in reflecting on important social characteristics and status of a group; and in social isolation, such as a perceived lack of social interaction.

And it is potentially the neurological basis of remembering the past and thinking about the future, including remembering the past; imagining the future; envisioning events that might happen in the future; episodic memory, that is, detailed memory related to specific events in time; story comprehension such as understanding and remembering a narrative; and replay, here meaning consolidating recently acquired memory traces.

Additionally, during attention demanding tasks, sufficient deactivation of the default mode network at the time of memory encoding has been shown to result in more successful long-term memory consolidation.

Studies have shown that when people watch a movie, listen to a story, or read a story, their DMNs are highly correlated with each other. DMNs are not correlated if the stories are scrambled or are in a language the person does not understand, suggesting that the network is highly involved in the comprehension and the subsequent memory formation of that story. The DMN is shown to even be correlated if the same story is presented to different people in different languages, further suggesting the DMN is truly involved in the comprehension aspect of the story and not the auditory or language aspect.

In accordance with embodiments disclosed herein, optimal targets within the nervous system are selected for neuromodulation. The optimal targets are selected according to network connectivity within the nervous system of a patient. For example, the brain of a patient may be modeled as a complex adaptive system of one or more neural networks. The brain may be viewed as exhibiting small world topology characteristics. That is, the brain functions as a modular scale free hierarchical network (e.g., fractal in organization). Also, the brain functions in the presence of noise (equivalently variability in neural activity) see, for example U.S. Pat. No. 8,682,441 in the field of pink noise therapy, the entire disclosure of which is incorporated herein by reference. In a noisy, hierarchical organization, the brain functions as a complex adaptive network of interconnected modules. By selecting one or more nodes within one or more networks within the brain for stimulation, a neurological disorder may be treated by strengthening or weakening the network connectivity, by controlling the phase of signals that pass along networks, or by creating correlated or anticorrelated connectivity communication relations between networks of interest to treat an identified neurological disorder.

Certain connectivity between neural populations in the brain may be defined by structural connectivity. The structural connectivity may be determined in studies using diffusion tensor imaging (DTI), diffusion spectrum imaging (DSI) or diffusion kurtosis imaging (DKI) as examples. Connectivity may also be the result of functional connectivity in a network. The functional connectivity may be determined by correlation or anticorrelation in neural activity in one or more respective brain areas or brain networks. Also, connectivity may be related to effective connectivity, which can be considered directional functional connectivity, through the result of information transfer between neural nodes and networks.

Structural connectivity refers to the presence of anatomical, biological-fiber pathways in the nervous system, which are relatively static at shorter time scales (seconds to minutes), but can be dynamic at longer time scales (hours to days) during learning or development. Thus, even anatomical connections are not hardwired but change with experience or deprivation thereof. Functional connectivity is fundamentally a statistical and not an anatomical concept, looking at patterns of correlated activity between different brain areas by measuring frequency or phase. In contrast to structural connectivity, which is based on hardwired anatomical white-matter tracts, functional connectivity changes constantly, by instantaneously adjusting correlated activity to endogenous or exogenous stimuli. Another form of functional connectivity computes cross-frequency coupling between different oscillatory frequencies, in which higher oscillations (beta and gamma) are nested in a hierarchical way on slower oscillatory frequencies (infraslow, slow, delta, theta and alpha) which act as carrier waves. Functional connectivity does not assume any directional flow of information. This is implicitly calculated by effective connectivity, which computes from where to where in the brain the information flows. Effective connectivity can, therefore, be considered directional, functional connectivity, and is often based on time series, where the underlying idea is that causes predate effects. Structural, functional and effective connectivity are all related to each other.

Functional connectivity is the basis of multiple separable brain networks, yet these brain networks are not all active at the same time. When one network is activated, others may be inactive or less active, resulting in anti-correlated activity between these networks. Some separable networks may be co-activated, leading to correlated activity. This has led to the development of the triple network model, which is a network-science based approach explaining core interactions in multiple cognitive and affective disorders. It states that neurological and psychiatric disorders are the result of aberrant interactions within and between three canonical brain networks. These three networks include the self-representational default mode network the behavioral relevance encoding salience network and the goal oriented frontoparietal central executive network. Normally, the salience network and the central executive network are characterized by correlated activity, and both networks are anti-correlated to the default mode network. The salience network acts as a switch between the anticorrelated default mode network and the central executive network. This is in keeping with the proposed functions of the three networks. When the salience network identifies a behaviorally relevant event in the environment, it reduces the activity of the self-oriented and mind wandering default mode network and activates the external goal-oriented central executive network to deal with the external salient event.

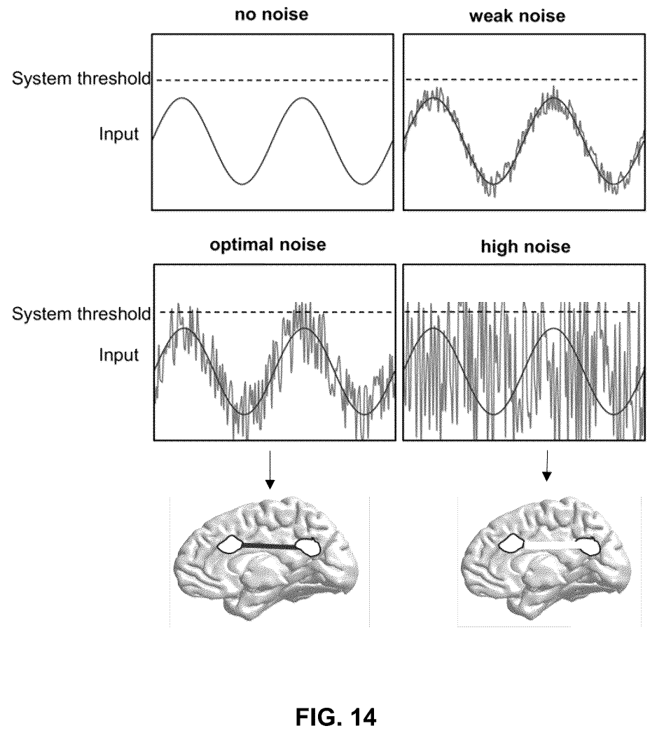

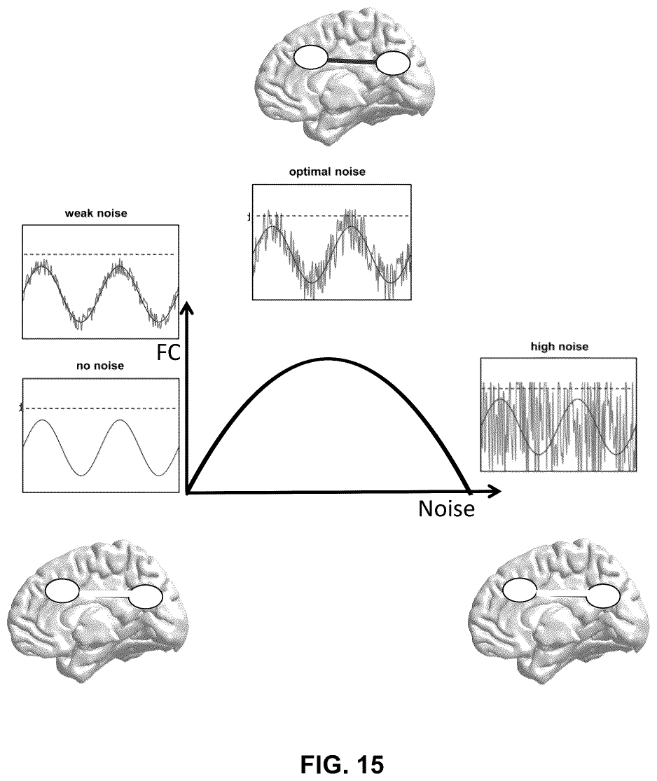

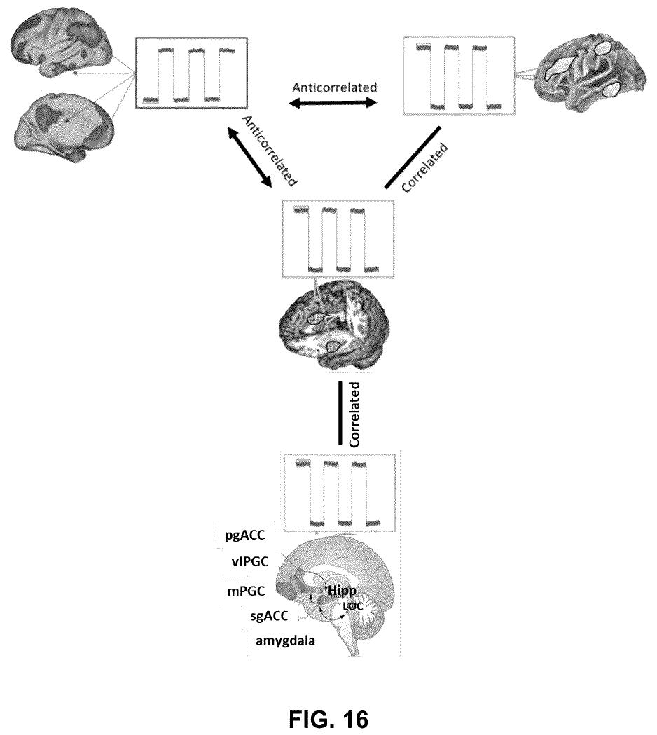

Functional and effective connectivity is constrained by the presence of both direct and indirect anatomical connections, and correlated activity can change structural connectivity via Hebbian mechanisms (cells that fire together wire together). These dynamical changes in structural, functional and effective connectivity are the basis of the concept of neuroplasticity and are crucial to developing novel devices that can not only break pathological connections but also rebuild normal physiological connections. It has been proposed that this requires two different stimulation designs, one that can optimally strengthen connectivity, such as a burst-like stimulation, and one that can break functional connections, such as a noise-like stimulation. Hyperconnectivity can be treated by surgical cutting of the connection or electrophysiologically by noise stimulation, low frequency stimulation in antiphase or pseudorandom burst stimulation. Hypoconnectivity on the other hand can be treated by burst stimulation in two targets in synchrony, by infraslow in phase stimulation and by noise stimulation. Noise stimulation can thus both break and build connectivity via a stochastic resonance effect (FIGS. 14, 15).

Multiple brain disorders exhibit similar changes in network activity and connectivity. These common pathophysiological mechanisms can be both genetic, physiological and anatomical. The same risk genes may cause multiple different neurological and psychiatric disorders, known as pleiotropy. Depending on the environment, the same risk genes may change functional connectivity, by modulating epigenetic gene expression in the brain, resulting in different emergent properties, i.e. different neurological and psychiatric disorders. For example, genetic overlap exists in the reward deficiency syndrome, a group of disorders encompassing addictions (substance and non-substance), impulsivity, obsessive compulsive and personality disorders with a common underlying mechanism.

Electrophysiologically, the entity called thalamocortical dysrhythmia groups pain, tinnitus, Parkinson's disease, depression and slow-wave epilepsy, and is characterized by a common core of beta activity in the dorsal anterior cingulate cortex (dACC) and the parahippocampus, and theta-gamma or theta-beta cross-frequency coupling in the respective motor or sensory cortex distinguishing the separate clinical entities.

Furthermore, many psychiatric disorders (schizophrenia, bipolar disorder, depression, addiction, obsessive-compulsive disorder, anxiety) share a common anatomical substrate. The salience-network dysfunction is at the core of these disorders. As the salience network, which is atrophic in many psychiatric disorders is dysfunctional, its function as a switch between internally directed cognition of the default mode network and externally directed cognition of the central executive network is disrupted. This leads to abnormal functional connectivity within and between these three networks as expressed by correlated and anti-correlated activity within and between the three cardinal networks. And indeed, common or shared hypo- and hyperconnectivity changes are identified in numerous brain disorders including ADHD, anxiety, depression, bipolar disorder, autism, OCD, PTSD and schizophrenia.

These anatomical and physiological shared mechanisms should permit the development of universal brain network neuromodulators that target the common pathophysiological mechanisms of these pathologies, rather than developing a dedicated device for each disorder individually.

As an example, triple network neuromodulation to treat ADHD, anxiety, depression, bipolar, autism, OCD, PTSD, or schizophrenia may involve sensing correlated activity within each of the 3 canonical networks, as well as between the 3 networks. If an abnormal infraslow phase synchronization is detected between some nodes, the nodes need to adjust their activity so that the intranetwork phase synchrony is restored, but also in such a way that the internetwork infraslow phase synchronies are restored to normal correlated activity between salience network and central executive network, and anticorrelated between these two networks and the default mode network. This may require the integrated activity of multiple distributed stimulators. Using network science, which studies complex adaptive systems, it has been shown that random attacks on (brain) networks are not capable of disrupting a network, and thus also not eliminating the emergent property of the network. Therefore, a targeted attack on the main hubs of the network or multiple interacting networks that are involved in the brain disorder is more likely to exert a beneficial effect. This is in agreement with a meta-analysis on deep brain stimulation for pain, which demonstrates that multitarget implants yield better outcomes than single-target stimulation, especially if both lateral and descending pain-inhibitory pathways are jointly targeted. Similarly, multitarget modulation also seems to be more beneficial for tinnitus than single-target stimulation.

The network nature of the brain may require that, if modulation is applied at multiple sites simultaneously, this modulation happens with a well-controlled degree of synchronicity so as to have the best possible (network) response and (patient) outcome. If the neuromodulation sites are covered by more than a single electrode, this requires careful orchestration of the timing of the stimulation.

Any number of suitable mechanisms may be employed to measure neuronal activity for suitable processing. For example, EEG (or electroencephalogram) is a recording of brainwave activity. QEEG (Quantitative EEG), popularly known as brain mapping, refers to a comprehensive analysis of brainwave frequency bandwidths that make up the raw EEG. QEEG is recorded the same way as EEG, but the data acquired in the recording are used to create topographic color-coded maps that show electrical activity of the cerebral cortex. In an QEEG analysis, the electrical activity of the brain is measured by placing a number of electrodes or sensors about the head of a patient and the sensors are connected to a recording device. Electrical activity is recorded using the sensors for typically ten to thirty minutes. The data representing the recorded electrical activity is suitably processed. The processing provides complex analysis of brainwave characteristics such as symmetry, phase, coherence, amplitude, power and dominant frequency. Such processing enables the correlation, coherence, and relevant activity metrics indicative of functional connection between brain locations to be identified. The analysis enables activity falling above or below a statistical norm to be identified for locations within the brain. Also, the activity may identify activity above or below the norm for relevant brainwave frequency bands (delta, theta, alpha, beta, and gamma bands as examples). The activity variance from the norm can be expressed relative to a calculated standard deviation of activity data. Further, the QEEG analysis additionally enables functional connectivity to be identified by coherence analysis of activity between different neural sites. The functional connectivity can be likewise expressed in terms of above or below the norm relative to a standard deviation calculation. Additional and/or alternative processing of recordings of electrical activity in the brain of a patient may be employed to assist identification of variations in functional connectivity related to a neurological disorder according to some embodiments. For example, QEEG combined with LORETA (Low Resolution Electromagnetic Tomography) enables examining of deep structures of the brain slice by slice, as well as viewing 3-dimensional models of the brain and may provide a suitable analysis to identify functional connectivity resulting from a neurological disorder to be treated according to representative embodiments. Also, the BrainWave® software application (available from the Department of Clinical Neurophysiology, VU University Medical Center, Amsterdam, The Netherlands) is an application for the analysis of multivariate neurophysiological data sets (such as EEG data sets). The BrainWave® application provides several measures of functional connectivity (coherence, phase coherence, imaginary coherence, PLI and synchronization likelihood) among other relevant neural activity metrics. The functional connectivity mapping of the BrainWave® application may be employed to assist identification of variations in functional connectivity related to a neurological disorder in a patient according to some embodiments.

In some embodiments, a network representation of neural activity is created using graph and network concepts.

Improper connectivity from a neurological disorder is addressed according to embodiments disclosed herein. In some embodiments, insufficient or decreased functional and/or effective connectivity related to a neurological disorder is strengthened by simultaneous or otherwise synchronized stimulation in two or more nodes of one or more neural networks relevant to the neurological disorder. In some embodiments, excessive functional and/or effective connectivity related to a neurological disorder is weakened by defective, malfunctioning, or unwanted correlated or anticorrelated communication connectivities between identified functional nodes or networks, along one or more neural pathways of a neural network relevant to the respective neurological disorder.

In a network connectivity framework, the centrality of a node refers to how many of the shortest paths between all other node pairs in a network pass through the respective node. As discussed herein, a hub refers to a network node in a neurological network which exhibits a high degree of centrality. Neurological hubs connect to many other brain areas. “Rich club” neurological sites refers to neurological sites that are hubs and are connected to many other hubs. Rich club sites integrate neurological activity from different networks and different neurological modules.

According to some embodiments, sites for neuromodulation are selected according to identified hubs (for example feeder hubs or hubs of the rich club or core). By selecting hub and rich club sites, improper connectivity (associated with a given neurological disorder) can be more effectively strengthened or weakened depending upon the specific neurological disorder.

Multiple types of stimulation patterns may be employed for network stimulation according to respective embodiments including tonic, burst, noise stimulation patterns, infraslow, slow and nested infraslow or slow patterns, as examples.

In some embodiments, nested stimulation may be provided to one or more nodes within one or more neural networks in association with adjusting connectivity within or between one or more networks. Details regarding design and generation of nested stimulation may also be found in U.S. Pat. No. 10,076,668 entitled “SYSTEM AND METHOD FOR NESTED NEUROSTIMULATION,” the entire disclosure of which is incorporated herein by reference.

Colors of Noise. Grey noise is a combination of pink noise and blue noise, or of brown noise and purple noise, or of black noise and blue noise, or of black noise and purple noise.

The relationships between categories of noise signals, their beta, or brainwave neural oscillation values, and real-world characterizations is given in the following two tables.

| | | | | |
|---|---|---|---|---|
| Color | Black | Brown, equal | Pink | White |
| | | to Red | | |
| 1/f^{β} | β > 2 | β = 2 | β = 1 | β = 0 |
| What | Natural | Random walk | Nature | Pure Noise |
| | disasters | | | |

| | | | | |
|---|---|---|---|---|
| Color | Blue, equal | Violet, equal | Grey | Green |
| | to Azure | to Purple | | |
| 1/f^{β} | −1 | −2 | U-Curve | Pink, with an |
| | | | | additional 500 Hz |
| What | Dithering | Acoustic | Perceived as | Background noise |
| | | thermal noise | equal | of world |

Firing Modes. Different firing modes or frequencies occur in the brain and/or other neuronal tissue, for example tonic firing and burst firing (including bath irregular or regular burst firing). The thalamus for example utilizes both types of firing modes. The two thalamni (bilateral paired structures) are the gateways to the cerebral cortex and, thus, to consciousness. The thalamic nuclei specialize in several different signaling functions: transmitting signals from sensory input to the cortex; transmitting signals from cortical motor centers to effectors; transmitting control signals that select which input and output will be permitted to pass to and from the cortex and how the signals will be sequenced (thalamic reticular nuclei (TRN)); modulated (controlling intensity), synchronized, or grouped (Intralaminar Nuclei (ILN)).

All thalamic relay neurons pass through the TRN, which opens and closes their “gates” going to the cortex, (McAlonan and Brown, 20(2). One mode that TRN neurons use to transmit these relays is burst firing mode. This mode is useful for activating a small population of neurons in the cortex for a short period. In contrast, the continuous (tonic) firing mode permits a thalamic neuron to transmit a steady stream of signals to the cortex. The tonic firing pattern triggers looping activation in the cortical circuits that receive the signals Evoking looping, or “recurrent” activation in the cortex requires a steady neural input.

Tonic or burst firing mode may be related to the molecules which are associated with the neurons. Such molecules include either parvalbumin (an egg-derived protein also a calcium-binding protein) or calbindin (a calcium-binding protein). Tonic firing is found especially in cells that contain parvalbumin. It behaves in a linear fashion, for example, the auditory thalamus (MGBV) fires at a specific frequency and the auditory cortex will follow at the same pace with a minor phase difference (Miller et al., 2001) of 2 ms. Tonic firing, however, can be overruled by burst firing (Lisman 1997; Sherman 2001; Swadlow and Gusev 2001).

Burst firing is typically found in calbindin positive cells (Kawaguchi and Kubota 1993; Hu et al., 1994; Hu 1995; He and Hu 2002). Thus, burst mode firing may utilize a calbindin system to generate the burst. Generally, burst firing is accomplished through the activation of either a subthreshold membrane conductance that initiates action potentials or a suprathreshold membrane conductance that once activated evokes two or more action potentials. Sodium (Na⁺) and calcium (Ca²⁺) activated conductances have all been implicated in burst generation.

Burst firing acts in a non-linear fashion with a summation effect of each spike, thus more readily activating a target cell than tonic firing can. Burst firing has been described in drowsiness, slow wave sleep, and anesthesia, as well as epilepsy in the thalamus. Neural network modeling has further demonstrated that bursts are generated by positive feedback through excitatory connections. In networks of two populations, one excitatory and one inhibitory, decreasing the inhibitory feedback can cause the network to switch from a tonically active, asynchronous state to the synchronized bursting state.

The generation of repetitive burst discharges in neurons is correlated with the generation of gamma frequency (30-70 Hz) oscillations in the local field potential. It is believed that conscious perception depends on gamma band frequency activity.

Increasing depolarization to hyperpolarization induces a prolonged refractory period of tonic tiring resulting in single spike bursts (for example in the visual system), and further depolarization results in progressively more spikes per burst. Further depolarization will temporarily silence the cell.

It is hypothesized according to the present invention that burst stimulation may be used by neuronal tissue to process information in a manner that is similar to amplitude modulation. Specifically, the spacing between individual bursts in a burst stimulus may be used to signal information to various regions in the brain. That is, the spacing between the bursts can vary (hence are “amplitude modulated”) to convey information. The signaled information can be related to relevance information. The signaled information could also be related to signaling the beginning and ending of certain packets of information. By providing electrical burst stimulation, it is possible that the stimulated brain tissue will change its processing of other stimulus information. For example, by appropriately selecting an interburst interval, auditory information that would otherwise be problematic to a patient could become ignored by a respective segment of the brain due to a lack of “relevance” and/or a lack of synchronization with the arrival of the burst stimulus.

Nested Stimulation and Electrical Stimulation Devices. Neurological stimulation (NS) systems, designated herein as pulse generators, execute the function of electrically stimulating a predetermined site area to treat one or more brain-related disorders or conditions as part of the Multi-Network Modulation therapeutic field of the present invention as claimed. In general terms, a pulse generator typically includes a pulse generating source and a non-invasive delivery system for delivering the stimulation waves to the brain. This can be contrasted with “implantable medical devices” or “IMDs”, which include components implanted into the patient's body and are not the subject of the current application. The delivery system may deliver a designed pattern of electrical pulses to a predetermined site. In certain preferred embodiments, the delivery system may include one or more delivery devices each coupled directly to the connecting portion of a stimulation lead.

In some embodiments, a delivery device is incorporated into a stimulation lead. For example, such a stimulation system may be commercially obtained as the Starstim stimulation system manufactured by Neuroelectrics or High Definition transcranial electrical stimulation devices commercialized by other companies such as Soterix Medical or Neuroconn GmbH. Whether the delivery device is coupled directly to, or embedded within, the stimulation lead, the delivery device controls the stimulation pulses transmitted to one or more stimulation electrodes that are located on the stimulating portion of a stimulation lead, and is positioned so as to be in communication with a predetermined site, according to suitable therapy parameters, for example noise color, correlation anticorrelation, phase coordination, phase non-coordination, duration, amplitude or intensity, frequency, pulse width, firing delay, and the like.

Suitable non-invasive stimulation methods may include any available transcranial stimulation method, including for example any one or more of the following.

Transcranial electrical stimulation (including high-definition transcranial electrical stimulation), using devices such as commercialized by Neuroelectrics, Soterix Medical, Neuroconn and other medical device companies. Transcranial electrical stimulation (tES) involves passing an electrical current through the brain. The electrical current is applied to an individual's scalp usually via two or more electrodes, and a portion of the current penetrates the scalp and is conducted through the brain. Withing the field of tES a number of different techniques are available, including transcranial direct current stimulation (tDCS), alternating current stimulation (tACS) and random noise stimulation (tRNS).

Transcranial Magnetic Stimulation (TMS) is a noninvasive neuromodulation technique where pulses of electromagnetic fields target and interact with specific brain regions from outside the head. There are several manufacturers of TMS systems, including Nexstim PLC, which uses a navigated transcranial magnetic stimulation (nTMS) technology called SmartFocus TMS in its TMS system. BrainsWay's proprietary Deep Transcranial Magnetic Stimulation (Deep TMS) platform technology directly stimulates deeper and broader areas of the brain. TMS devices are widely used in inpatient and outpatient psychiatric facilities. The invention can be applied to rTMS if systems are developed that can target multiple brain areas simultaneously, or when multiple single-target devices are used simultaneously.

Transcranial ultrasound stimulation applies ultrasound waves of approximately 500-650 kHZ to the brain. Ultrasound is defined as a sound or acoustic wave higher than 20 kHz (i.e., above human hearing). This is also known as Low Intensity Focused Ultrasound or LIFU. The invention can be applied to LIFU if systems are developed that can target multiple brain areas simultaneously, or when multiple single-target devices are used simultaneously.

Transcranial light stimulation, aka as photobiomodulation applies infrared frequencies (650-1200 nM) to the brain, penetrating the scalp and skull without heating. It is aka as low-level laser therapy (LLLT), cold laser therapy, or red light therapy applies low-level (low-power) lasers or light-emitting diodes (LEDs) to the surface of the brain. The invention can be applied to photobiomodulation if systems are developed that can target multiple brain areas simultaneously, or when multiple single-target devices are used simultaneously.

As contemplated in embodiments herein, a predetermined stimulation site for tissue of interest can include either peripheral neuronal tissue and/or central neuronal tissue. Neuronal tissue includes any tissue associated with the peripheral nervous system or the central nervous system. Peripheral neuronal tissue can include a nerve root or root ganglion or any neuronal tissue that lies outside the brain, brainstem or spinal cord. Peripheral nerves can include, but are not limited to olfactory nerve, optic, nerve, oculomotor nerve, trochlear nerve, trigeminal nerve, abducens nerve, facial nerve, vestibulocochlear (auditory) nerve, glossopharyngeal nerve, vagal nerve, accessory nerve, hypoglossal nerve, suboccipital nerve, the greater occipital nerve, the lesser occipital nerve, the greater auricular nerve, the lesser auricular nerve, the phrenic nerve, brachial plexus, radial axillary nerves, musculocutaneous nerves, radial nerves, ulnar nerves, median nerves, intercostal nerves, lumbosacral plexus, sciatic nerves, common peroneal nerve, tibial nerves, sural nerves, femoral nerves, gluteal nerves, thoracic spinal nerves, obturator nerves, digital nerves, pudendal nerves, plantar nerves, saphenous nerves, ilioinguinal nerves, genitofemoral nerves, and iliohypogastric nerves.

Central neuronal tissue includes brain tissue, spinal tissue or brainstem tissue. Brain tissue can include thalamus/sub-thalamus, basal ganglia, hippocampus, amygdala, hypothalamus, mammillary bodies, substantia nigra or cortex or white matter tracts afferent to or efferent from the abovementioned brain tissue, inclusive of the corpus callosum. Spinal tissue can include the ascending and descending tracts of the spinal cord, more specifically, the ascending tracts of that comprise intralaminar neurons or the dorsal column. The brainstem tissue can include the medulla oblongata, pons or mesencephalon, more particular the posterior pons or posterior mesencephalon, Lushka's foramen, and ventrolateral part of the medulla oblongata.

A doctor, the patient, or another user may directly or indirectly input or program designed therapy parameters to specify or modify the nature of the stimulation provided.

The selected stimulation system can, for example, be programmed by the therapy designer to apply any desired stimulation wave or combination of stimulation waves, including e.g. infraslow or slow stimulation waves or nested stimulation, particularly where the electrical pulses are in the waveform of a chosen color, for example pink noise, that is nested upon an electrical pulse in the waveform of an infraslow or slow carrier wave, or it can for example alternatively by programmed to apply a burst-type electrical pulse stimulation to targeted brain tissue of a patient, or to apply burst stimulation sequentially combined with noise stimulation in any of the noise stimulation waveforms that may be used in therapy. Specifically, the stimulation system may include a microprocessor and a pulse generation module. The pulse generation module generates the electrical pulses according to a defined pulse width and pulse amplitude and applies the electrical pulses to defined electrodes. The microprocessor controls the operations of the pulse generation module according to software instructions stored in the device.

A stimulation system can be adapted by programming its internal microprocessor to deliver a number of spikes (relatively short pulse width pulses) that are separated by an appropriate interspike interval. Thereafter, the programming of the microprocessor causes the pulse generation module to cease pulse generation operations for an interburst interval. The programming of the microprocessor also causes a repetition of the spike generation and cessation of operations for a predetermined number of times. After the predetermined number of repetitions has been completed within a nested stimulation waveform, the programmed microprocessor can cause burst stimulation to cease for an amount of time (and resume thereafter). Also, in some embodiments, the microprocessor could be programmed to cause the pulse generation module to deliver a hyperpolarizing pulse before the first spike of each group of multiple spikes.

The microprocessor can be programmed to allow the various characteristics of the burst stimulus to be set by a physician to allow the burst stimulus to be optimized for a particular pathology of a patient. For example, the spike amplitude, the interspike interval, the interburst interval, the number of bursts to be repeated in succession, the electrode combinations, the firing delay between nested stimulation waveforms delivered to different electrode combinations, the amplitude of the hyperpolarizing pulse, and other such characteristics could be controlled using respective parameters accessed by the microprocessor during burst stimulus operations. These parameters could be set to desired values by an external programming device via wireless communication with the neuromodulation device.

In alternative representative embodiments, a stimulation system may apply electrical stimulation according to a suitable noise signal by which is meant white noise, pink noise, purple noise, blue noise, grey noise, brown noise, black noise, or green noise. Details regarding implementation of a suitable noise signal can be found in U.S. Pat. No. 8,682,441, the entire disclosure of which is incorporated herein by reference.

In another embodiment, a stimulation system may be implemented to apply burst stimulation using a digital signal processor and one or several digital-to-analog converters. The burst stimulus waveform could be defined in memory and applied to the digital-to-analog converter(s) for application through electrodes of the medical lead. The digital signal processor could scale the various portions of the waveform in amplitude and within the time domain (e.g., for the various intervals) according to the various burst parameters.

A nested stimulation (NS) system may be controlled to deliver various types of nested stimulation therapy, such as high frequency neurostimulation therapies, burst neurostimulation therapies and the like. High frequency neurostimulation includes a continuous series of monophasic or biphasic pulses that are delivered at a predetermined frequency, most preferably color noise stimulation waveform frequency patterns nested upon a low frequency waveform and designed so as to produce a reinstated normal anticorrelation connectivity or communication between networks, for example the brain's central executive network and the brain's default node network. Burst neurostimulation however includes short sequences of monophasic or biphasic pulses, where each sequence is separated by a quiescent period.

The NS system may deliver nested stimulation therapy based on preprogrammed therapy parameters. The therapy parameters may include, among other things, pulse amplitude, pulse polarity, pulse width, pulse frequency, interpulse interval, inter-burst interval, electrode combinations, firing delay and the like. Optionally, the NS system may represent a closed loop neurostimulation device that is configured to provide real-time sensing functions from a lead. The configuration of the lead sensing electrodes may be varied depending on the neuronal anatomy of the sensing site(s) of interest. The size and shape of electrodes is varied based on the electrode location. The electronic components within an NS system may be designed with both stimulation and sensing capabilities, including alternative nested stimulation therapy, such as burst mode, high frequency mode and the like.

An NS system may include a delivery device that is adapted to generate designed electrical pulses for application to tissue of a patient. The delivery device may include any suitable components, e.g. any one or more of: a housing or can that encloses a controller, a pulse generating circuit, a charge storage circuit, a battery, a far-field and/or near field communication circuit, a battery charging circuit, a switching circuit, a memory circuit, and the like. A suitable charge storage circuit may represent one or more capacitors and/or battery cells that store charge used to produce the therapies described herein. The pulse generating circuitry, under control of the controller, manages discharge of the charge storage circuit in order to shape the morphology of the waveform delivered while discharging energy. The switching circuitry thus connects select combinations of the device's electrodes a pulse generating circuitry, thereby directing the stimulation waveform to a desired electrode combination. As explained herein, such switching circuitry successively connects the pulse generating circuitry to any variety of successive electrode combinations. A controller typically includes one or more processors, such as a microcontroller, for controlling the various other components of the device. Software code is typically stored in memory for execution by the microcontroller or processor to control the various components of the device. A delivery device may comprise a separate or an attached extension component. If the extension component is a separate, discrete component, the extension component may connect with the “header” portion of the delivery device, as it is known in the art. If the extension component is integrated with the delivery device, internal electrical connections may be made through respective conductive components. Within the delivery device, electrical pulses are generated by a suitable pulse generating circuit and are provided to a switching circuitry. A switching circuitry connects to outputs of the delivery device. Electrical connectors (e.g., “Bal-Seal” connectors) within the connector portion of an extension component, or within the delivery device header may be employed to conduct various stimulation pulses. The terminals of one or more leads are inserted within a connector portion or within the delivery device header for electrical connection with respective connectors. In this fashion, the pulses originating from the delivery device are provided to the lead. Any pulses generated are then conducted through the conductors of a lead and applied to selected tissue of a patient via conventional stimulation electrodes that are coupled to blocking capacitors. Any suitable known or later developed design may be employed for a connector portion.

Stimulation electrodes may be positioned along a horizontal axis of a lead, and may be, for example, angularly positioned about the horizontal axis, so that the stimulation electrodes aren't arrayed so as to overlap. Adjacent stimulation electrodes may be separated from one another by non-conducting rings that may electrically isolate each stimulation electrode from any adjacent stimulation electrode. Such non-conducting rings may include one or more insulative materials and/or biocompatible materials. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane. Stimulation electrodes may be configured to emit their pulses in a suitable direction proximate to or towards a stimulation target. The stimulation electrodes may deliver noise, tonic, high frequency and/or burst nested stimulation waveforms as described herein. Optionally, the electrodes may usefully also sense neural oscillations and/or sensory action potential (neural oscillation signals) for a data collection, identification and/or recordation window in near-simultaneous time, enabling a suitably programmed device to generate waveforms as desired by the clinician to be applied to the patient in response to the sense oscillations detected and identified.

A lead may comprise a lead body of insulative material about one or more conductors within the insulative material that extend from a proximal end of the lead, proximate to the transcranial stimulator, to its distal end. Conductors electrically couple a plurality of the stimulation electrodes to a plurality of terminals of the lead. Terminals are adapted to receive electrical pulses, and stimulation electrodes may be adapted to apply generated pulses to the stimulation target of the patient. Also, sensing of physiological signals may occur through stimulation electrodes, conductors, and terminals. Although not required for any particular embodiments, the lead body of a lead may be fabricated to flex and/or elongate for accommodating movements of the patient.

Different burst and/or noise high frequency pulses nested on low frequency pulses on different stimulation electrodes may be generated using a single set of pulse generating circuitry using consecutively generated pulses according to a “multi-stimset program” as they are known in the art, or by using multiple sets of pulse generating circuitry. Complex pulse parameters may be employed such as those described in U.S. Pat. No. 7,228,179, entitled “Method And Apparatus For Providing Complex Tissue Stimulation Patterns”, and International Patent Publication Number WO2001/093953 A1, entitled “Neuromodulation Therapy System,” the entire written and illustrative disclosures of each of them being incorporated herein by reference. Alternatively, multiple sets of such circuitry may be employed to provide such varied pulse patterns. Various sets of parameters may define the pulse characteristics and pulse timing for the pulses applied to various stimulation electrodes. Although constant current pulse generating circuitry is contemplated for some embodiments, any other suitable type of pulse generating circuitry may be employed such as constant voltage pulse generating circuitry.

A controller may deliver a stimulation wave (e.g. a nested stimulation waveform) to at least one electrode combination located proximate to nervous tissue of interest, the nested stimulation waveform including a series of pulses configured to excite, for example nested C-fibers of nervous tissue of interest, the nested stimulation waveform and the carrier waveform defined by therapy parameters or preprogrammed therapy parameters that are based upon information collected from numerous past patients, patient clinical trials, medical literature, and/or tests performed upon an individual patient.

In simultaneous signal recordation and stimulation, a controller can sense intrinsic neural oscillations from at least one of the electrodes on a lead. The controller may analyze such intrinsic neural oscillations signals to obtain useable brain activity data. Then, the controller can determine whether the collected (and recorded) activity data satisfies some selected criteria of interest i.e. normal or baseline functionality. The controller then adjusts at least one of the therapy parameters to change the carrier and nested stimulation waveform or waveforms selected, when the activity data does not satisfy the criteria of interest. The controller then iteratively repeats such delivering operations for a group of TPS. The controller selects a candidate TPS from the group of TPS known to be based on criteria of interest. The controller may repeat the delivering, sensing and adjusting operations to optimize the stimulation wave. The analyzing operation may include analyzing a feature of interest from a morphology of the neural oscillation signal over time, counting a number of occurrences of the feature of interest that occur within the signal over a predetermined duration, and the generation of activity data based on the number of occurrences of the feature of interest.

Memory stores software configured to control operation of a controller for nested stimulation therapy as explained herein. The memory may also store neural oscillation signals, therapy parameters, neural oscillation activity level data, sensation scales and the like. For example, the memory may save neural oscillation activity level data for various different therapies as applied over a short or extended period of time. A collection of neural oscillation activity level data is accumulated for different therapies and may be compared to identify high, low and acceptable amounts of sensory activity.

A controller device may be implemented to manage the processes of the charge and recharge a suitable battery of any device in the system (although a separate recharging device could alternatively be employed) and to program pulse specifications. In alternative embodiments, separate programmer devices may be employed for charging, recharging and/or programming. The controller device selected may be a processor-based system that possesses wireless communication capabilities. Software may be stored within a non-transitory memory of the controller device which may be executed by the processor to control the various operations of the controller device. A controller device may provide one or more user interfaces (e.g., a touchscreen, a keyboard, a mouse, one or more buttons, or the like) allowing the user to operate the delivery of stimulation waves. The controller device may be controlled by the user (e.g., physician, nurse, therapist) through the user interface, thereby allowing the user to interact with the delivery device. The user interface of choice may permit the user to move electrical stimulation along and/or across one or more lead(s) using different stimulation electrode combinations, for example, as described in U.S. Patent Application Publication No. 2009/0326608A1, entitled “Method Of Electrically Stimulating Tissue Of A Patient By Shifting A Locus Of Stimulation And System Employing The Same,” the entire written and illustrative disclosure of which is incorporated herein by reference.

Alternatively, a controller device may permit operation of the delivery device according to one or more therapies to treat the patient. Each therapy may include one or more sets of stimulation parameters of the pulse including pulse amplitude, pulse width, pulse frequency or inter-pulse period, firing delay, pulse repetition parameter (e.g., number of times for a given pulse to be repeated for respective stimset during execution of program), biphasic pulses, monophasic pulses, etc. In this fashion, the delivery device modifies its internal parameters in response to the control signals it receives from its controller device to vary the stimulation characteristics of stimulation waves transmitted to the targeted tissue(s) of the patient. NS systems, stimulation headsets (stimsets), and different stimset programs are discussed in PCT Publication No. WO2001/093953A1, entitled “Neuromodulation Therapy System,” and U.S. Pat. No. 7,228,179, entitled “Method And Apparatus For Providing Complex Tissue Stimulation Patterns,” which are expressly incorporated herein by reference.

For percutaneous use, a percutaneous stimulation lead can include one or more circumferential-shaped electrodes spaced apart from one another along the length of a stimulating portion of a stimulation lead. Circumferential electrodes emit electrical stimulation energy in a substantially radial emission pattern, that is generally perpendicular to the axis of a stimulation lead, in all directions. A laminotomy, paddle, or surgical stimulation lead can include one or more directional stimulation electrodes spaced apart from one another along one surface of a stimulation lead. Although various types of stimulation leads are described herein as examples, a given embodiment of a stimulation system may include any suitable type of stimulation lead in any suitable number, and stimulation leads may be used alone or in combination. For example, medial or unilateral stimulation of the predetermined site may be accomplished using a single electrical stimulation lead in communication with the predetermined site of interest in one side of the head, while bilateral electrical stimulation of the predetermined site may be accomplished using two stimulation leads in communication with the predetermined site in opposite sides of the head.

A delivery device can allow each electrode of each lead to be defined as having a positive, a negative, or a neutral polarity. For each electrode combination (e.g., the defined polarity of at least two electrodes having at least one cathode and at least one anode), an electrical signal can have at least a definable amplitude (e.g., voltage), pulse width, and frequency, where these variables may be independently adjusted to finely select the sensory transmitting brain tissue required to inhibit transmission of neuronal signals. Generally, amplitudes, pulse widths, and frequencies are determinable by the capabilities of the neurostimulation systems, which are known by those of skill in the art. Voltages that may be used can include, for example about 0.5 to about 10 volts, more preferably about 1 to about 10 volts.

In different embodiments herein, the therapy parameter of signal frequency may be varied to achieve a burst type rhythm, or burst mode stimulation. Generally, the burst stimulus frequency may be in the range of about 0.01 Hz to about 100 Hz, more particularly, in the range of about 1 Hz to about 12 Hz, and still more particularly, in the range of about 1 Hz to about 4 Hz, 4 Hz to about 7 Hz or about 8 Hz to about 12 Hz for each burst. Each burst stimulus comprises at least two spikes, for example, each burst stimulus can comprise about 2 to about 100 spikes, more particularly, about 2 to about 10 spikes. Each spike can comprise a frequency in the range of about 50 Hz to about 1000 Hz, more particularly, in the range of about 200 Hz to about 500 Hz. The frequency for each spike within a burst can be variable, thus it is not necessary for each spike to contain similar frequencies, e.g., the frequencies can vary in each spike. The inter-spike interval can also vary, for example, the inter-spike interval, can be about 0.1 milliseconds to about 100 milliseconds or any range there between. A burst stimulus is followed by an inter-burst interval, during which substantially no stimulus is applied. The inter-burst interval may have a duration in the range of about 1 millisecond to about 5 seconds, more preferably, about 10 milliseconds to about 300 milliseconds. A burst stimulus may be configured to have a duration in the range of: about 1 millisecond to about 5 seconds; in the range of about 250 msec to 1000 msec (1-4 Hz burst firing); in the range of 145 msec to about 250 msec (4-7 Hz); in the range of 145 msec to about 80 msec (8-12 Hz); or in the range of 1 to 5 seconds in plateau potential firing. The burst stimulus and the inter-burst interval can have a regular pattern or an irregular pattern (e.g., random or irregular harmonics). More specifically, the burst stimulus can have a physiological pattern or a pathological pattern. Additional details regarding burst stimulation and stimulus patterns may be found in U.S. Pat. No. 8,897,870, the entire written and illustrative disclosure of which is incorporated herein by reference.

It is envisaged that the patient will require intermittent assessment with regard to successful or beneficial therapeutic patterns of stimulation. Different electrodes on the lead can be selected by suitable computer programming, such as that described in U.S. Pat. No. 5,938,690, the entire disclosure of which is incorporated herein by reference. The use of such a program may allow for an optimal stimulation pattern to be obtained at minimal voltages. This additionally ensures a longer battery life for implanted systems. Example fabrication processes are disclosed in U.S. Pat. No. 9,054,436, entitled, “Method Of Fabricating Stimulation Lead For Applying Electrical Stimulation To Tissue Of A Patient,” the entire written and illustrative disclosure of which is incorporated herein by reference.

Nesting on Infraslow or Slow Electrical Signals. One or more neurological signal noises may be nested on infraslow or slow electrical signals, in order to normalize selected target communications and hence connectivity, within and in between disrupted brain networks, in order to treat neurological and/or psychological and/or psychiatric conditions and/or disorders.

Neurostimulation (NS) systems are devices that generate electrical pulses and deliver the pulses to nervous tissue to treat a variety of disorders. The NS device may comprise one or more electric pulse generators or other suitable stimulation source. An example of their use is deep brain stimulation, which has been used to treat movement disorders such as pain, Parkinson's disease and affective disorders such as depression. Recently, new stimulation configurations such as burst stimulation and high frequency stimulation, have been developed, in which closely spaced high frequency pulses are delivered. In general, conventional neurostimulation systems seek to manage pain and other pathologic or physiologic disorders through stimulation of select nerve fibers that carry pain related signals. However, nerve fibers and brain tissue carry other types of signals, not simply pain related signals. Although some neurological disorders have been treated through known neurostimulation methods, many other neurological disorders exhibit physiological complexity, functional complexity, or other complexity and have not been adequately treated through known neurostimulation methods. Consequently, no novel method of neurostimulation can be predicted to be safe and efficacious without carefully designed preclinical and clinical studies to observe the results. Hence novel neuromodulatory therapeutics cannot genuinely be found to be obvious to a practitioner of ordinary skill in this art without such testing and observation.

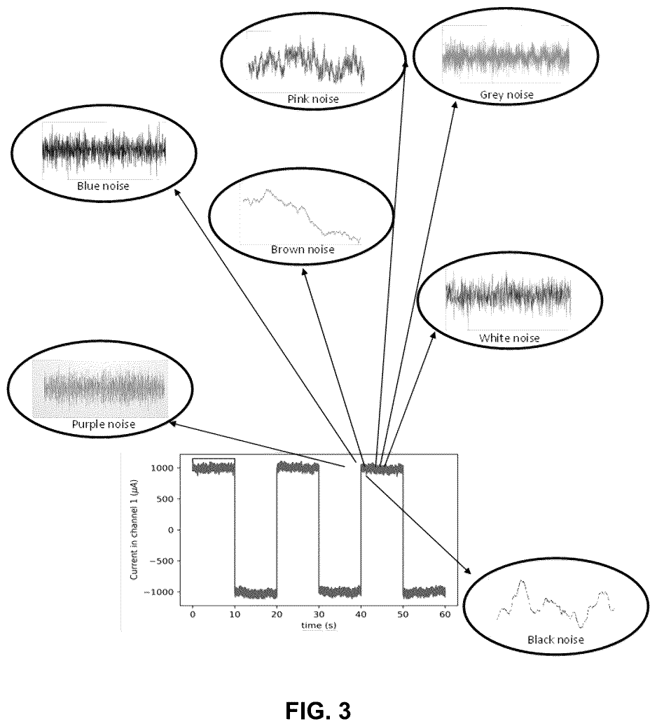

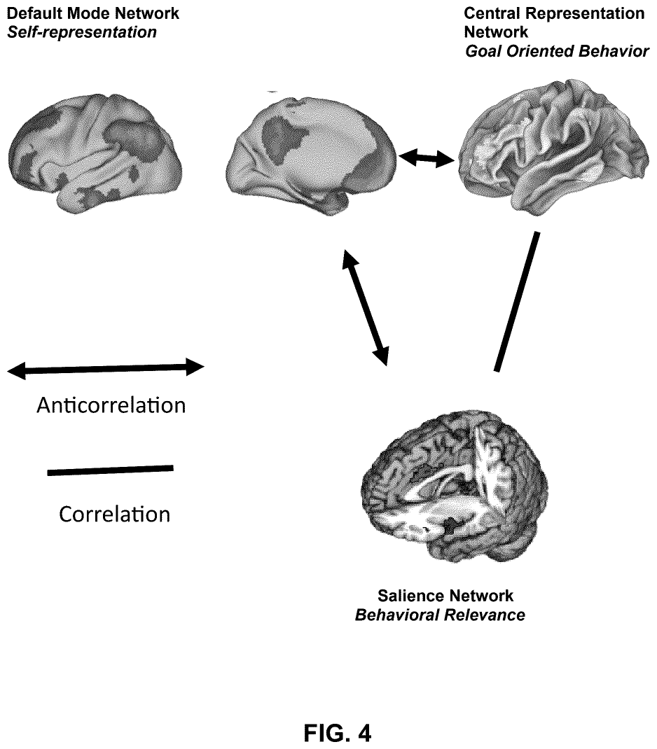

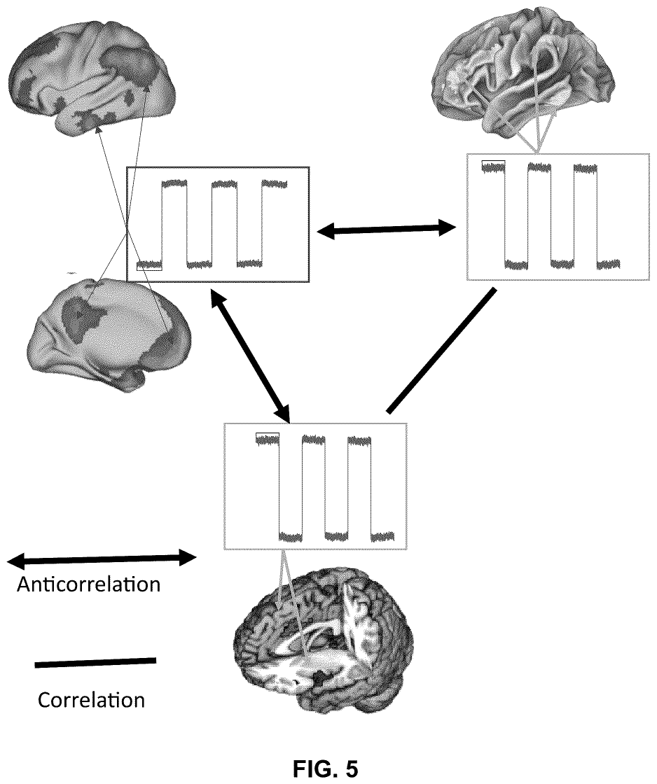

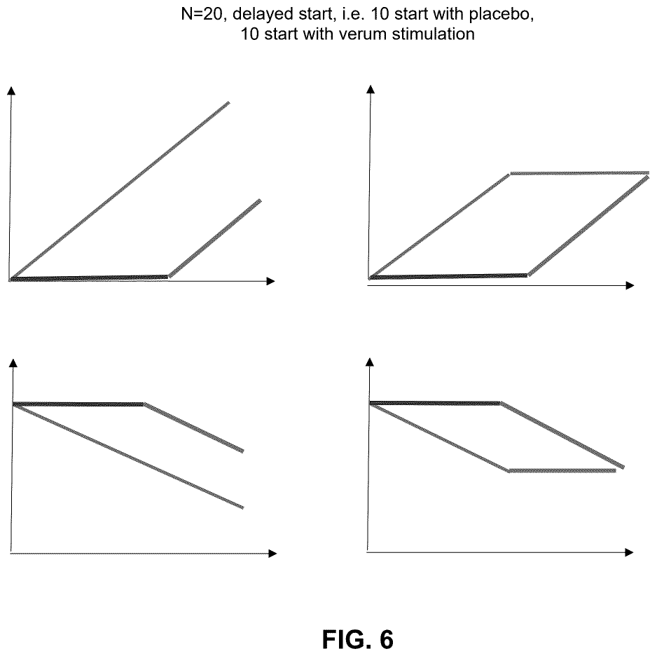

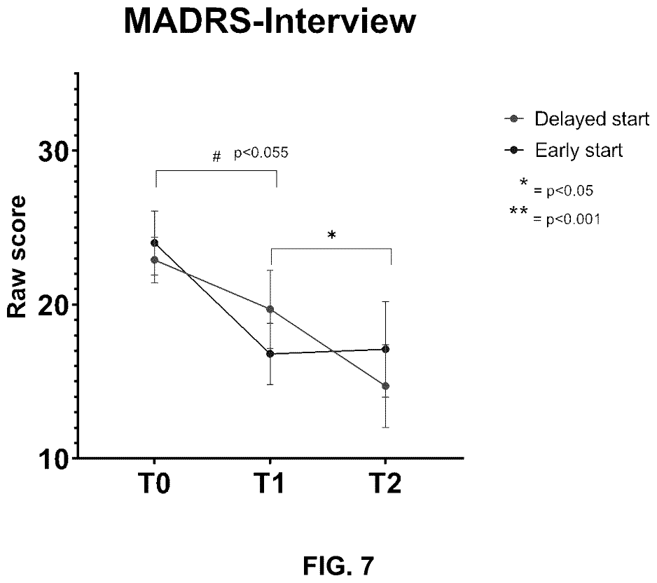

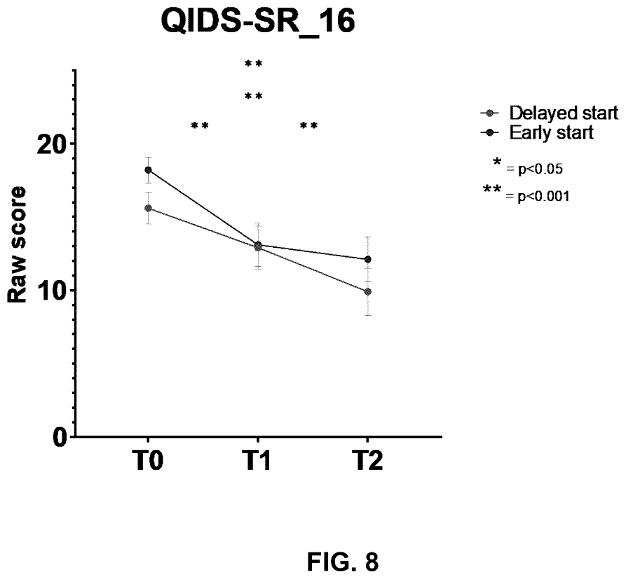

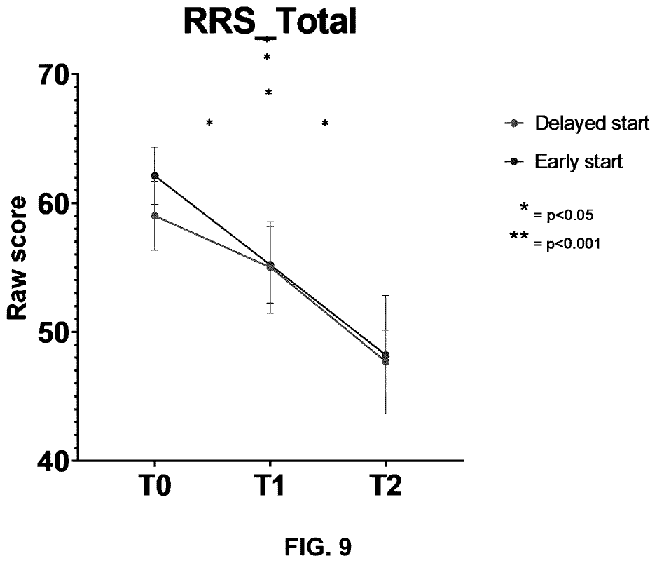

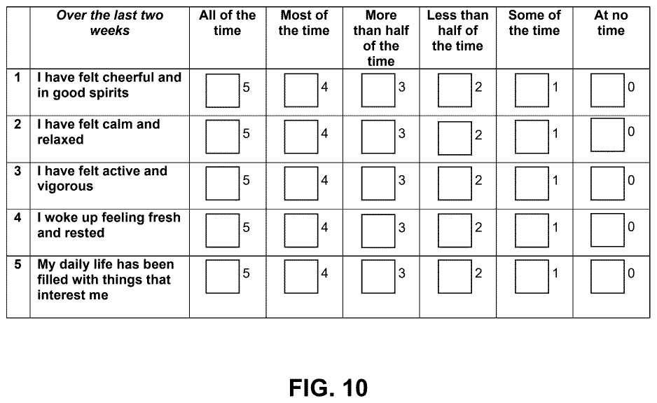

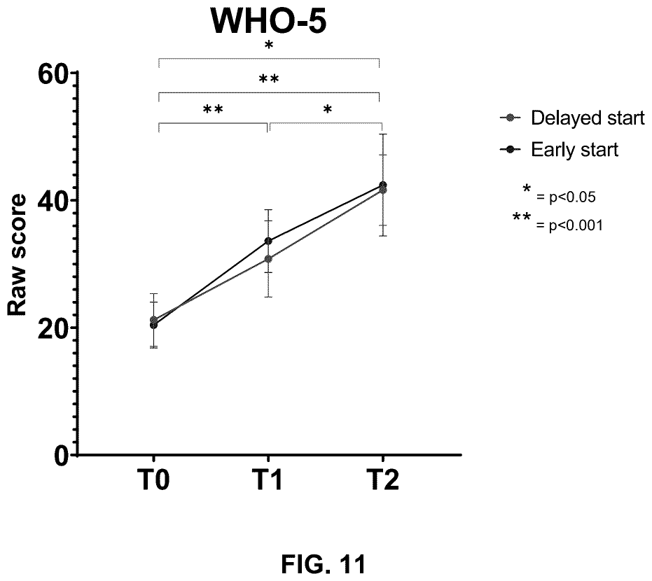

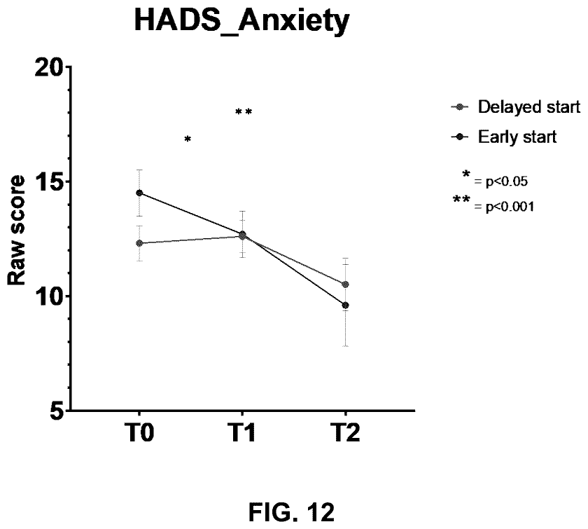

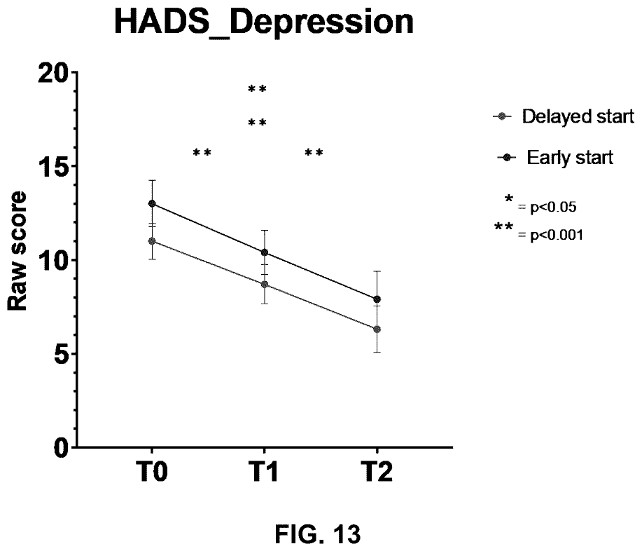

Turning to FIG. 3, there is shown a drawing of oscilloscopic waveforms that illustrate the relationship of relatively high frequency waves that are nested upon an infraslow or slow carrier wave shown here as a function of electrical current versus time. At the upper end of one waveform burst, here in the range of approximately 1,000 microAmperes (p A), seven different classes of signal noise (colors) are shown as alternative colors that may be selected as the stimulation of choice, being nested upon the carrier wave. Specifically, there are shown in FIG. 3 the noise colors Blue, Purple, Grey, Black, Pink, Brown, Black, and White. It is to be understood that although an exemplary wave form for Green Noise is not illustrated here, that Green Noise is another noise that may be selected in the stimulation design.

The Applicant's system therefore aims to ameliorate abnormal behaviour in brain networks by applying infraslow or slow activity to a number of brain networks. This may be done in such a way that infraslow or slow stimulation is correlated between two or more networks, and/or anti-correlated between two or more networks. For example, infraslow or slow stimulation may be correlated between areas of the salience and central executive networks, and anticorrelated between those networks and the default mode network. In this example, normal correlated activity between the salience and central executive networks may be promoted, strengthened or restored, and normal anticorrelated activity between those networks and the default mode network may be promoted, strengthened or restored. Abnormal or pathological activity may be reduced, disrupted or ameliorated.

Further, Applicant's methods may be used to achieve amelioration of a brain-state thereby providing a neuroenhancement. For example, ameliorating a brain state may involve ameliorating a non-pathological brain state in order to achieve an improvement in cognition, improved resilience, improved performance, improved sports performance or any other amelioration, enhancement or improvement in brain function.

The Applicant's methods may be used with any suitable combination of brain networks where amelioration of a brain state involving those networks provides a desirable outcome.

In addition to networks discussed elsewhere in this specification, other brain networks that may be acted upon include but are not limited to the ventral attention network (inferior parietal, ventrolateral prefrontal), dorsal attention network (premotor, superior parietal), memory network, emotion/affective network, emotional network (amygdala, subgenual anterior cingulate cortex, orbitofrontal cortex), mirror neuron network (ventrolateral prefrontal, inferior parietal, superior temporal sulcus), auditory network, visual network, somatosensory network, vestibular network, motor network, central autonomic nervous system network, central autonomic (control) network (insula, amygdala, anterior cingulate, posterior cingulate/precuneus, brainstem, hypothalamus, mediodorsal thalamus, periaqueductal grey, ventral tegmental area), sensorimotor network (visual, auditory, somatosensory, vestibular, motor), speech network (Wernicke and Broca areas) or any other suitable brain network known in the field. Further, other brain regions may be acted upon. Noise signals, such as pink, brown, blue, purple or grey noise, or any suitable combination thereof, may be superimposed on the infraslow or slow stimulation wave.

In some embodiments, only an infraslow or slow stimulation wave may be used, free of nested signals.

Applicant's methods may be used for treating any suitable condition that involves an abnormal interaction between brain regions. This may include any suitable brain-related disorder, including any suitable neurological, psychological or psychiatric condition. For example, Applicant's methods may be used in treatment of: tinnitus, epilepsy, depression, anxiety, Parkinson's Disease, autonomic dysfunctions (including cardiac, respiratory, cardiorespiratory, urogenital, gastrointestinal disorders), immune disorders (e.g. via innervation of spleen and bone), stress, attention deficit hyperactivity disorder, bipolar disorder, autism, obsessive compulsive disorder, post-traumatic stress disorder syndrome, or schizophrenia, as well as mild cognitive impairment, dementias (Alzheimer's disease, Lewy-body dementia, multi-infarct) but also in thalamocortical dysrhythmias (tinnitus, pain, Parkinsons disease), epilepsy, and disorders of consciousness (minimally cognitive state, vegetative state/unresponsive wakefulness syndrome).

In general, any suitable non-invasive stimulation method may be used, including e.g. transcranial magnetic stimulation, transcranial electrical stimulation, transcranial ultrasound, or transcranial optic (light or laser) stimulation, in all their different forms. Currently, tES may be preferred.

In some embodiments, a plurality of energy sources or stimulation channels may be used. For example, at least six, or between 6 and 32, independently controlled energy sources or stimulation channels may be used. In some embodiments up to 128 independently controlled energy sources or stimulation channels may be used. Different stimulation waves (having e.g. one or more of: different carrier amplitude, different carrier frequency, different carrier pulse width, different nested signals, different nested frequency etc) may be directed to different brain regions.

The amplitude of the stimulation wave may be in the range 0.01 mA to 15 mA.

The pulse width of the nested signal may be in the range 10 microseconds to 5 milliseconds, with a frequency up to around 1000 Hz. The nested signal may be a burst or tonic signal.

The Applicant's method may be applied to any desired number of brain networks. For example, the method may be used with three networks (triple network neuromodulation) as in some of the examples described above. In further embodiments the method may be applied to four (quadruple network neuromodulation), five (quintuple network neuromodulation) or a higher number of brain networks.

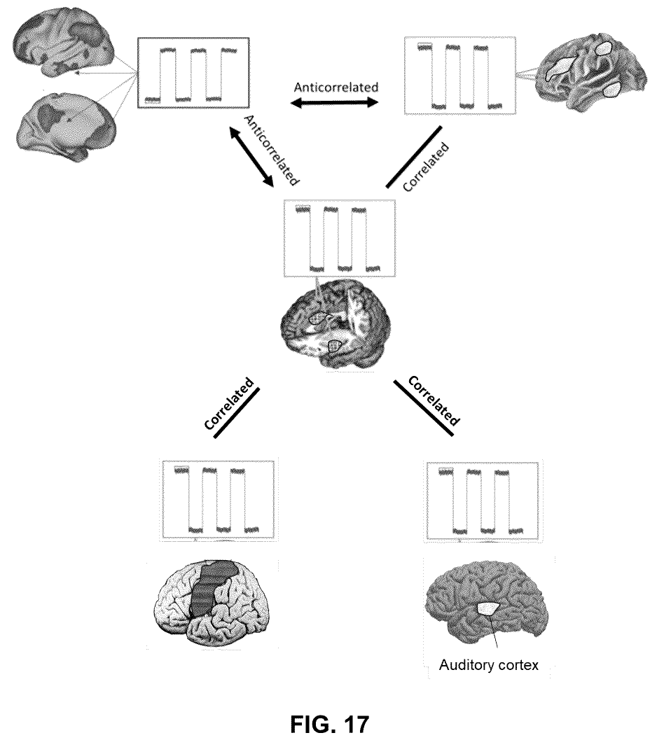

As shown in FIG. 17, adding a fourth network including the auditory cortex may allow treatment of tinnitus. Adding a fourth network including the somatosensory network may allow treatment of pain.

Adding a fourth network including the emotional network may allow treatment of depression and/or anxiety.

A five network stimulation arrangement may allow treatment of further conditions. For example, adding the somatosensory and emotional networks may allow treatment of pain and depression.

Adding the emotional and ventral attention networks may allow treatment of schizophrenia.

The skilled reader will understand that Applicant's methods may be employed with various combinations of brain networks for treatment of different conditions or amelioration of different brain states.

Further, sensors may be provided to detect brain activity. Sensors may be separate components connected through a separate sensing system. Alternatively, sensors may be attached to the neurostimulation system. Further, the stimulators or delivery devices and sensors may be provided in one unit or even through the same components. For example, in one embodiment sensing and stimulation may be provided through the same electrodes of an eTS system.

Sensing of brain activity (e.g. sensing of correlated and/or anticorrelated activity) may be performed before neurostimulation, for example for diagnostic purposes. Sensed data may be sent to a controller or processor and may be recorded or stored in memory. A controller or processor may compare the sensed or recorded data to a look up table, database or other diagnostic record. Once a brain state is diagnosed, neurostimulation characteristics may be determined automatically by the controller or processor, or may be set by a user. Neurostimulation characteristics may include, for example, any one or more of: target brain network, node, amplitude, frequency, phase of the infraslow or slow stimulation wave, amplitude, frequency, phase of a high frequency nested signal, noise type for nested signal, identifiers of particular electrodes or delivery devices etc.

Sensing may also detect correlated and/or anticorrelated activity during treatment. For example, periods of sensing may be interspersed with periods of stimulation. This allows the system to function in a closed loop fashion via automated adjustment of the neurostimulation characteristics and stimulation design.

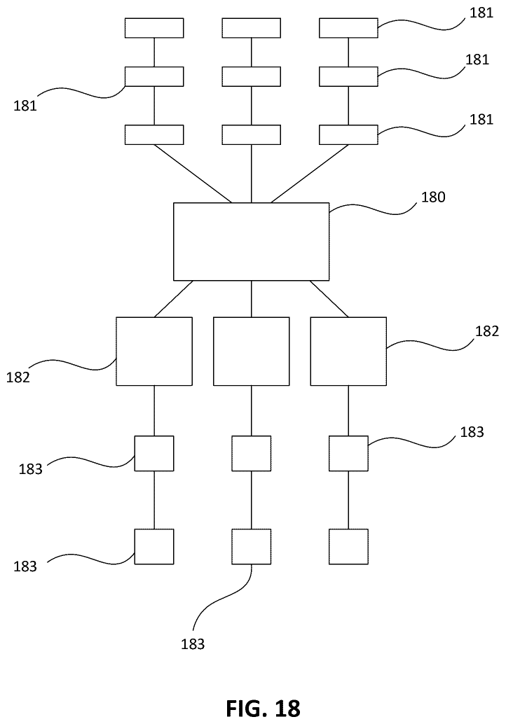

FIG. 18 is a schematic drawing showing one example of a system in which a controller 180 receives sensor information from a number of sensors 181. The controller controls a number of stimulation wave generators 182, which provide stimulation waves to a number of delivery devices (e.g. electrodes) 183. The skilled reader will understand that alternative system architectures or structures are possible.

Based on the studies illustrated in the attached drawings, Applicant's system appears to have beneficial effect in treating depression, with patients generally trending from severe or moderate depression to no or mild depression over time. Anxiety also appears to respond well. More treatment sessions appear to yield better results. The effect of Applicant's methods is distinguished from the placebo effect.

It is to be understood that the subject matter described herein is not limited in its application to the details of patient diagnosis and the design of therapeutic intervention for that patient as set forth in the described disclosure herein or illustrated in the drawings hereof. The subject matter described herein is capable of other methodological embodiments and of being practiced or of being carried out in various ways. The discussion above is based on Applicant's current understanding of the science and it is not intended to be bound by theory. Also, it is to be understood that the phraseology and terminology used herein is for the purpose of description and should not be regarded as limiting. The use of “including,” “comprising,” or “having” and variations thereof herein is meant to encompass the anatomy and functionality of neurological networks and nodes, the histological tissues, and items listed thereafter and equivalents thereof as well as additional items. Furthermore, it is to be understood that the above description is intended to be illustrative, and not restrictive. For example, the above-described embodiments (and/or aspects thereof) may be applied in combination with each other. In addition, many modifications may be made to adapt a particular situation or therapeutic design to the teachings of the invention without departing from its scope. While the types of apparatus described herein are intended to define the electric signal or current output neuromodulatory parameters of the invention, they and their operational output are by no means limiting and are exemplary embodiments. Many other embodiments will be apparent to those of skill in the art after reviewing and learning the above described disclosure. The scope of the invention should, therefore, be determined with reference to the appended claims, along with the full scope of equivalents to which such claims are entitled. In the appended claims, the terms “including” and “in which” may be used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 45 U.S.C. § 112(f), unless and until such claim limitations expressly use the phrase “means for” followed by a statement of function void of further structure.

1-27. (canceled)

28. A non-invasive method of ameliorating a brain-related state in a patient, the method including ameliorating one or more interactions between a first brain network and one or more further brain networks by:
delivering a first infraslow or slow stimulation wave to the first brain network; and
delivering a second infraslow or slow stimulation wave to at least one of the one or more further brain networks;
wherein a phase difference between the first and second infraslow or slow stimulation waves is selected for amelioration of the brain-related state.

29. The method as claimed in claim 28, further including ameliorating an abnormal condition within the first brain network.

30. The method as claimed in claim 28, including delivering one or more further infraslow or slow stimulation waves to one or more of the further brain networks.

31. The method as claimed in claim 28, wherein at least one of the first and second stimulation waves includes a high frequency waveform nested upon an infraslow or slow carrier waveform.

32. The method as claimed in claim 31, wherein the high frequency waveform includes one or more of: pink noise, brown noise, red noise, black noise, grey noise, white noise, blue noise, violet noise, or green noise.

33. The method as claimed in claim 31, wherein the high frequency waveform is generated in a pseudo-random manner.

34. The method as claimed in claim 31, including setting high-frequency waveform parameters that define the high frequency waveform and wherein at least one of said carrier waveform and said high frequency waveform is defined to correspond to physiologic neural oscillations associated with at least one of: said first brain network and said one or more further brain networks.

35. The method as claimed in any claim 28, wherein said first brain network and one or more further brain networks include the central executive network, the default node network and the salience network.

36. The method as claimed in claim 35, wherein the one or more further brain networks include a fourth network selected from the ventral attention network, dorsal attention network, memory network, emotion/affective network, emotional network, mirror neuron network, auditory network, visual network, somatosensory network, vestibular network, motor network, central autonomic nervous system network, central autonomic (control) network, sensorimotor network, and speech network.

37. The method as claimed in claim 35, wherein the one or more further brain networks include a fourth and fifth network selected from: ventral attention network, dorsal attention network, memory network, emotion/affective network, emotional network, mirror neuron network, auditory network, visual network, somatosensory network, vestibular network, motor network, central autonomic nervous system network, central autonomic (control) network, sensorimotor network, and speech network.

38. The method as claimed in any claim 28, including: determining the presence of an abnormal correlation interaction or an abnormal anticorrelation interaction between the first brain network and at least one of the one or more further brain networks.

39. The method as claimed in claim 28, including: recording electrical activity related to neural activity in the brain of said patient; and, with reference to said recording, determining the presence of an abnormal correlation interaction or an abnormal anticorrelation interaction between the first brain network and at least one of the one or more further brain networks.

40. The method as claimed in claim 28, including:
setting first waveform parameters that define the first infraslow or slow stimulation wave; and
setting second waveform parameters that define the second infraslow or slow stimulation wave.

41. The method as claimed in claim 28, including: providing one or more stimulation wave generators configured to generate the first and second stimulation waves.

42. A method as claimed in claim 28, wherein said abnormal interactions include one or more abnormal correlation interactions and/or one or more abnormal anticorrelation interactions.

43. The method as claimed in claim 28, wherein ameliorating one or more abnormal interactions includes reinstating a normal correlated interaction.

44. The method as claimed in claim 28, wherein ameliorating one or more abnormal interactions includes reinstating a normal anticorrelated interaction.

45. The method as claimed in claim 28, wherein said all stimulation waveforms are delivered to said patient contemporaneously.

46. The method as claimed in claim 28, wherein said brain-related disorder or brain-related state is characteristic of one or more of: disease attention deficit hyperactivity disorder, anxiety, depression, bipolar disorder, autism, obsessive compulsive disorder, post-traumatic stress disorder syndrome, or schizophrenia as well as mild cognitive impairment, dementias (Alzheimer, Lewy-body disease, multi-infarct) but also in thalamocortical dysrhythmias (tinnitus, pain, Parkinson Disease), stress, epilepsy, and disorders of consciousness (minimally cognitive state, vegetative state/unresponsive wakefulness syndrome), stress autonomic nervous system disorders including immune disorders.

47. A non-invasive neuromodulation system arranged to deliver stimulation waves trans-cranially to the brain of a patient, the system including:
a first stimulation wave delivery unit, arranged to deliver a first infraslow or slow stimulation wave to a first brain network of the patient; and
one or more further stimulation wave delivery units, each arranged to deliver a further infraslow or slow stimulation wave to a further brain network of the patient; and
a controller arranged to control a phase difference between the first and second infraslow or slow stimulation waves, in order to ameliorate an abnormal interaction between the first brain network and at least one of the further brain networks.
